(19) United States
(12) Patent Application Publication
(10) Pub. No.: US 2011/0269812 A1
(43) Pub. Date: Nov. 3, 2011

(54) NOVEL BENZYLIDENE-INDOLINONE AND THEIR MEDICAL AND DIAGNOSTIC USES

(75) Inventors: Mei Lin Go, Singapore (SG); Han Kiat Ho, Singapore (SG); Wei Zhang, Singapore (SG)

(73) Assignees: Agency for Science, Technology and Research, Singapore (SG); National University of Singapore, Singapore (SG)

(21) Appl. No.: 13/124,112

(22) PCT Filed: Oct. 14, 2009
(86) PCT No.: PCT/SG2009/000376

§ 371 Date: Jul. 12, 2011

Related U.S. Application Data
(60) Provisional application No. 61/105,206, filed on Oct. 14, 2008.
(60) Provisional application No. 61/145,595, filed on Jan. 19, 2009.

(51) Int. Cl. A61K 31/404; A61P 35/00; C12Q 1/18; C07D 209/34; C12N 9/99

(52) U.S. Cl. 514/418; 548/486; 435/184; 435/32

(57) ABSTRACT

The present invention relates generally to organic chemistry, biochemistry, pharmacology and medicine. More particularly, it relates to 3-benzylidene-indolin-2-one derivatives and their physiologically acceptable salts and prodrugs which modulate the activity of protein kinases (“PKs”), especially protein tyrosine kinase, and, therefore, are expected to exhibit a salutary effect against disorders related to abnormal PK activity. The present invention is further directed to methods of using of these compounds, alone or in combination with other therapeutic agents, for the alleviation, prevention and/or treatment of protein kinase-mediated diseases and disorders, such as cancer.

### CROSS-REFERENCE TO RELATED APPLICATIONS

This application claims the benefit of priority of U.S. provisional application No. 61/105,206, filed Oct. 14, 2008, and U.S. provisional application No. 61/145,595, filed Jan. 19, 2009, the contents of both applications being hereby incorporated by reference in its entirety for all purposes.

### FIELD OF THE INVENTION

The present invention relates generally to organic chemistry, biochemistry, pharmacology and medicine. More particularly, it relates to 3-benzylidene-indolin-2-one derivatives and their physiologically acceptable salts and prodrugs which modulate the activity of protein kinases (“PKs”), especially protein tyrosine kinases, and, therefore, are expected to exhibit a salutary effect against disorders related to abnormal PK activity. The present invention is further directed to methods of using of these compounds, alone or in combination with other therapeutic agents, for the alleviation, prevention and/or treatment of protein kinase-mediated diseases and disorders, such as cancer.

### BACKGROUND OF THE INVENTION

The use of tyrosine kinase inhibitors (TKIs) for treating diseases related to unregulated protein kinase signal transduction have been subject to extensive research over the past two decades. For example, the use of indolinone compounds useful for the treatment of diseases including cell proliferative diseases such as cancer, atherosclerosis, arthritis and restenonsis and metabolic diseases such as diabetes was described in U.S. Pat. No. 6,147,106.

TKIs are generally important pharmacological agents in the growing field of targeted therapy against tyrosine kinase related diseases including cancers. Conventionally, the mechanistic basis for their application hinges on monogenetic lesions such as Bcr-Abl translocation in chronic myelogenic leukemia or HER2 amplification in breast carcinomas, which provide exclusive signal for driving tumorigenesis—i.e. oncogene addiction (Weinstein IB. Cancer. Addiction to oncogenes—the Achilles heal of cancer. Science 2002 Jul. 5; 297(5578):63-64). However, mounting evidences suggest that kinase mutations for many malignancies tend to be sporadic rather than congregating on specific “hotspots” in the genome (Bleeker F E, Bardelli A. Genomic landscapes of cancers: prospects for targeted therapies. Pharmacogenomics 2007 December; 8(12):1629-1633; Greenman C, Stephens P, Smith R, Dalgliesh G L, Hunter C, Bignell G, et al. Patterns of somatic mutation in human cancer genomes. Nature 2007 Mar. 8; 446(7132):153-158; Ruhe J E, Streit S, Hart S, Wong C H, Specht K, Knyazev P, et al. Genetic alterations in the tyrosine kinase transcriptome of human cancer cell lines. Cancer Res 2007 Dec. 1; 67(23):11368-11376; Thomas R K, Baker A C, Debiasi R M, Winckler W, Laframboise T, Lin W M, et al. High-throughput oncogene mutation profiling in human cancer. Nat Genet 2007 March; 39(3):347-351). This implies that the underlying driver for disease initiation and progression likely involves multiple oncogenic defects which undermine the effectiveness of monotherapy. Moreover, recent studies also demonstrated that receptor cross-talk is pervasive and provides the means for tumors to switch oncogenic dependence. For example, MET amplification was described in non-small cell lung carcinoma resistant to EGFR inhibition (Engelman J A, Zejnullahu K, Mitsudomi T, Song Y, Hyland C, Park J O, et al. MET amplification leads to gefitinib resistance in lung cancer by activating ERBB3 signaling. Science 2007 May 18; 316(5827):1039-1043); IGF-1R signaling was also upregulated to circumvent growth arrest caused by erlotinib (Buck E, Eyzaguirre A, Rosenfeld-Franklin M, Thomson S, Mulvihill M, Barr S, et al. Feedback mechanisms promote cooperativity for small molecule inhibitors of epidermal and insulin-like growth factor receptors. Cancer Res 2008 Oct. 15; 68(20):8322-8332). As a result, targeting multiple kinases presumably reduces the incidence of alternative kinase usage which leads to chemoresistance in chronic therapy (Petrelli A, Giordano S. From single- to multi-target drugs in cancer therapy: when aspecificity becomes an advantage. Curr Med Chem 2008; 15(5):422-432; Weinstein I B, Joe A. Oncogene addiction. Cancer Res 2008 May 1; 68(9):3077-3080).

Hepatocellular carcinoma (HCC) exemplifies a cancer where an undisputable disease-causing activating mutation in cell-signaling remains elusive. Several tyrosine kinases have been reported to associate with HCC development and growth. For instance, overexpression of FAK, PYK2, IGF-1R and FGFR3 were reported separately and might contribute to disease severity (Itoh S, Maeda T, Shimada M, Aishima S, Shirabe K, Tanaka S, et al. Role of expression of focal adhesion kinase in progression of hepatocellular carcinoma. Clin Cancer Res 2004 Apr. 15; 10(8):2812-2817; Qiu W H, Zhou B S, Chu P G, Chen W G, Chung C, Shih J, et al. Over-expression of fibroblast growth factor receptor 3 in human hepatocellular carcinoma. World J Gastroenterol 2005 Sep. 14; 11(34):5266-5272; Scharf J G, Braulke T. The role of the IGF axis in hepatocarcinogenesis. Horm Metab Res 2003 November; 35(11-12):685-693; Sun C K, Ng K T, Sun B S, Ho J W, Lee T K, Ng I, et al. The significance of proline-rich tyrosine kinase2 (Pyk2) on hepatocellular carcinoma progression and recurrence. Br J Cancer 2007 Jul. 2; 97(1):50-57). Most recently, the elevation of FGFR4 in about one-third of HCC patients in normal-to-tumor transition was reported and its potential link to AFP regulation was established (Ho H K, Pok S, Streit S, Ruhe J E, Hart S, Lim K S, et al. Fibroblast growth factor receptor 4 regulates proliferation, anti-apoptosis and alpha-fetoprotein secretion during hepatocellular carcinoma progression and represents a potential target for therapeutic intervention. J Hepatol 2009 January; 50(1):118-127). Currently, clinical trials of TKIs in HCC employ empirical usage of approved or investigative drugs that were initially designed for other diseases. Not surprisingly, these trials yielded marginal effects because they were not necessarily targeting the specific tyrosine kinases implicated in the disease. For instance, phase II clinical studies with imatinib and erlotinib in unresectable HCC showed disappointing responses when the majority of patients recruited showed disease progression and none experienced complete or even partial response (Lin A Y, Fisher G A, So S, Tang C, Levitt L. Phase II study of imatinib in unresectable hepatocellular carcinoma. Am J Clin Oncol 2008 February; 31(1):84-88; Thomas M B, Chadha R, Glover K, Wang X, Morris J, Brown T, et al. Phase 2 study of erlotinib in patients with unresectable hepatocellular carcinoma. Cancer 2007 Sep. 1; 110(5):1059-1067).

Although sorafenib (Nexavar®), a multi-targeting TKI with submicromolar inhibitory effects towards VEGFRs, PDGFRs, RET, c-kit as well as Raf kinase, was approved by FDA for HCC treatment in 2007 (Simpson D, Keating G M. Sorafenib: in hepatocellular carcinoma. Drugs 2008; 68(2):251-258) and trials with another multi-targeting TKI, sunitinib, are presently ongoing (Zhu A X. Development of sorafenib and other molecularly targeted agents in hepatocellular carcinoma. Cancer 2008 Jan. 15; 112(2):250-259), there remains need in the art for potent broad spectrum TKIs with directed efficacy against cancer.

This need is solved by compounds of the present invention and their pharmaceutical uses.

### SUMMARY OF THE INVENTION

The present invention is directed to 3-benzylidene-indolin-2-one derivatives and their physiologically acceptable salts and prodrugs which modulate the activity of protein kinases (“PKs”) and, therefore, are expected to exhibit a salutary effect against disorders related to abnormal PK activity, such as cancer.

In a first aspect, the invention is directed to a compound of formula I

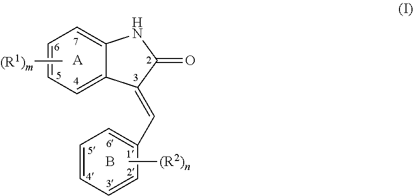

wherein:
each R¹ and R² is independently selected from the group consisting of unsubstituted or substituted C₁-C₁₀ alkyl, unsubstituted or substituted C₂-C₁₀ alkenyl, unsubstituted or substituted C₂-C₁₀ alkynyl, unsubstituted or substituted C₁-C₁₀ alkoxy, hydroxyl, halo, and trihalomethyl;
m is an integer 1 and n is an integer 1 or 2;
if each of m and n is 1, then R¹ is at position 6 of ring A and R² is at position 2′, 3′ or 4′ of ring B; and
if m is 1 and n is 2, then R¹ is at position 6 of ring A and R² are at positions 3′ and 4′ of ring B;
or a pharmaceutically acceptable salt or prodrug thereof.

In another aspect, the invention is directed to a compound of formula II:

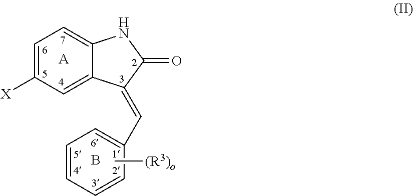

wherein:
X is F, Cl, Br or C₁-C₄ alkoxy;
each R³ is independently selected from the group consisting of unsubstituted or substituted C₁-C₁₀ alkyl, unsubstituted or substituted C₂-C₁₀ alkenyl, unsubstituted or substituted C₂-C₁₀ alkynyl, unsubstituted or substituted C₁-C₁₀ alkoxy, hydroxyl, halo, and trihalomethyl;
o is an integer 1 or 2;
if o is 1, then R³ is at position 3′ or 4′ of ring B;
if o is 2, then R³ are at positions 3′ and 4′ of ring B;
or a pharmaceutically acceptable salt or prodrug thereof.

In a further aspect, the invention is directed to a method of preparing a compound of formula I or II, the method comprising reacting an oxindole of formula (III)

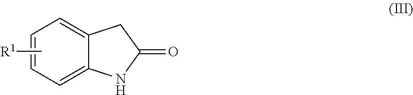

with an aldehyde of formula (IV)

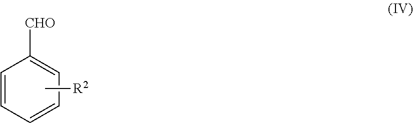

in the presence of a base.

In still another aspect, the invention is directed to a pharmaceutical composition comprising a compound of the invention or salt or prodrug thereof and a pharmaceutically acceptable carrier or excipient.

In another aspect, the present invention is directed to a method for the modulation of the catalytic activity of a protein kinase comprising contacting said protein kinase with at least one compound, salt or prodrug according to the invention.

In still another aspect, the present invention relates to a method for the treatment or prevention of protein tyrosine kinase-related disease or disorder, comprising the administration of a pharmaceutically active amount of a compound according to the invention to a subject in need thereof.

In still another aspect, the invention is directed to a method of identifying a protein kinase inhibitor having specific efficacy against hepatocellular carcinoma, comprising:
(i) incubating a candidate compound with a hepatocellular carcinoma cell line;
(ii) determining cell viability; and
(iii) comparing the determined cell viability with the candidate compound's effects on a normal liver cell line to identify a compound with specific activity against hepatocellular carcinoma cells

The present invention is further described in the following detailed description and with reference to the following brief description of the drawings.

### BRIEF DESCRIPTION OF THE DRAWINGS

FIGS. 1A and 1B show the fluorescence-activated cell sorting (FACS) diagrams showing the effects of two illustrative compounds of the invention compounds 46 and 48 on HCT116 cells released from (FIG. 1A) G1-block and (FIG. 1B) G2-block. R6, R3, R4, R5 refer to sub-G1, G1, S and G2/M phases respectively. Compounds 46 and 48 were tested at 15 μM and 5 μM respectively (≈1.5×IC₅₀).

FIG. 2 shows the viability of an illustrative compound of the invention, compound 47 vs. sunitinib (HepG2, HuH7 and THLE2). (A) HuH7, (B) HepG2 and (C) THLE2 cells were plated on 96-well format and treated with 0-10 μM Compound 47 (▪) and sunitinib () for 72 h. Viability was measured by Cell-Titer Glo and results expressed as a percentage of viable cells in the untreated controls with +/− coefficient of variation (n=8).

FIG. 3 shows the effects of an illustrative compound of the invention, compound 47 on separate panel of HCC cell lines. The efficacy of compound 47 against HCC proliferation was tested on a wider panel of HCC cell lines: Hep3B (), Hs817T (▪), PLC/PRF/5 (▴) and SK-Hep 1 (▾), as determined by Cell Titer-Glo assay.

FIG. 4 shows a Western blot analysis of an illustrative compound of the invention, compound 47 in HepG2 and HuH7. HuH7 or HepG2 cells were treated with increasing concentration of Compound 47 (0, 1, 5 or 10 μM) for 24 h. Cell lysates were harvested and immunoblotted against cleaved PARP, phospho-Erk, phospho-Akt, PCNA, cyclin D1, Bax and Bcl-xL, with HSP60 as loading control.

FIG. 5 shows the effects of an illustrative compound of the invention, compound 47 on caspase-3 activity in HuH7 and HepG2. HuH7 (darker bar) and HepG2 (lighter bar) cells treated as described in FIG. 4 were harvested for caspase-3 assay performed by the catalytic hydrolysis of fluorogenic Ac-DEVD-AMC. Results were expressed as a normalized RFU per μg lysate per h of incubation with the AMC substrate (n=3).

FIG. 6 shows that an illustrative compound of the invention, compound 47, potently inhibits AFP transcription in HuH7. HuH7 cells were treated with Compound 47 (1, 5, 10 μM) or sunitinib (10 μM) for 24 h. Real-time PCR was performed AFP. The resulting average CT values were normalized against 18S mRNA as housekeeping control and data was expressed as a fold change in transcript expression vs. untreated controls. Error bars represent standard deviation converted to fold changes (n=3).

FIG. 7 shows an RTK array and the effects of an illustrative compound of the invention compound 47 on RTK phosphorylation. Serum starved HuH7 cells were treated with Compound 47 (1, 5, 10 μM) or sunitinib (10 μM) for 24 h. Kinase phosphorylation changes were determined with human phospho-RTK array.

FIG. 8 shows the effects of an illustrative compound of the invention, compound 47 on IGF-1R, EGFR, EphA2 and Tyro3 phosphorylation by immunoblot analysis. (A) HuH7 cells were treated with vehicle, Compound 47 (1, 5 or 10 μM) or sunitinib (10 μM) for 24 h. Samples were immunoblotted against phospho-IGF-1R (Y980 and Y1135/1136).

Loading control was with HSP60. (B) Cells treated similarly were immunoprecipitated with anti-EGFR, anti-EphA2 and anti-Tyro3 before immunoblotting with anti-phosphotyrosine (4G10). Membranes were subsequently reprobed with respective antibodies as loading controls.

### DETAILED DESCRIPTION OF THE INVENTION

The present invention provides a compound of formula I:

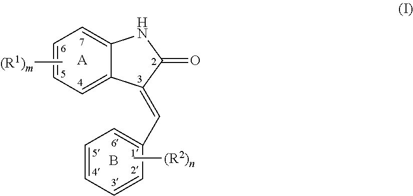

In the compound of formula I or a pharmaceutically acceptable salt or prodrug thereof, each of the substituents R¹ and R² is independently selected from the group consisting of unsubstituted or substituted C₁-C₁₀ alkyl, unsubstituted or substituted C₂-C₁₀ alkenyl, unsubstituted or substituted C₂-C₁₀ alkynyl, unsubstituted or substituted C₁-C₁₀ alkoxy, hydroxyl, halo, and trihalomethyl. The integer m has a value of 1 and the integer n has a value of 1 or 2. Thus, if each of m and n is 1, then R¹ is at position 6 of ring A and R² is at position 2′, 3′ or 4′ of ring B. If m is 1 and n is 2, then R¹ is at position 6 of ring A and R² are at positions 3′ and 4′ of ring B.

In one embodiment of the compound of formula I, each R¹ is independently selected from the group consisting of halo and C₁-C₄ alkoxy. Hence, in certain embodiments, R¹ is independently selected from the group consisting of bromo, chloro, fluoro and methoxy.

In another embodiment of the compound of formula I, each R² is independently selected from the group consisting of C₁-C₄ alkoxy, hydroxyl and trihalomethyl. In certain embodiments, each R² is independently selected from the group consisting of methoxy, ethoxy, hydroxy, and trifluoromethyl.

In some embodiments, the invention is directed to a compound of formula II:

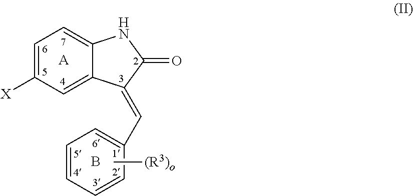

In the compound of formula (II) or a pharmaceutically acceptable salt or prodrug thereof, the substituent X is F, Cl, Br or C₁-C₄ alkoxy. Each of the R³ substituent is independently selected from the group consisting of unsubstituted or substituted C₁-C₁₀ alkyl, unsubstituted or substituted C₂-C₁₀ alkenyl, unsubstituted or substituted C₂-C₁₀ alkynyl, unsubstituted or substituted C₁-C₁₀ alkoxy, hydroxyl, halo, and trihalomethyl. The integer o has a value of 1 or 2. Thus, if o is 1, then R³ is at position 3′ or 4′ of ring B. If o is 2, then R³ are at positions 3′ and 4′ of ring B.

In one embodiment of the compound of formula II, each R³ is independently selected from the group consisting of C₁-C₄ alkoxy, hydroxy and trihalomethyl. In certain embodiments, each R³ is independently selected from the group consisting of methoxy, hydroxyl and trifluoromethyl.

In another embodiment of the compound of formula II, X can be methoxy or ethoxy.

The compounds as described herein possess potent antiproliferative activity and are particularly useful in treating diseases and disorders that are connected to an inappropriate or abnormal protein tyrosine kinase function, in particular increased protein tyrosine kinase activity, or that involve protein tyrosine kinase function. Diseases and disorders that may thus be treated and/or prevented by the compounds of the present invention are by way of example, without being limited to these diseases and disorders, hyperproliferative disorders and cancers, such as hepatocellular carcinoma, lung cancer, colon cancer and breast cancer. At the same time, the inventors have found that the compounds of the invention also provide chemopreventive potential (see Example 5). Thus, the compounds as described herein are not only expected to exhibit a salutary effect against disorders related to abnormal PK activity such as cancer but have potential cytoprotection towards normal cells.

As used herein, “Alkyl” refers to a saturated aliphatic hydrocarbon including straight chain, or branched chain groups. Preferably, the alkyl group has 1 to 10 carbon atoms (whenever a numerical range; e.g., “1-10”, is stated herein, it means that the group, in this case the alkyl group, may contain 1 carbon atom, 2 carbon atoms, 3 carbon atoms, 4 carbon atoms, 5 carbon atoms, 6 carbon atoms etc. up to and including 10 carbon atoms). More specifically, it may be a medium size alkyl having 1 to 6 carbon atoms or a lower alkyl having 1 to 4 carbon atoms e.g., methyl, ethyl, n-propyl, isopropyl, butyl, iso-butyl, tert-butyl and the like. The alkyl group may be substituted or unsubstituted. When substituted, the substituent group(s) is one or more, for example one or two groups, individually selected from the group consisting of C₃-C₈ cycloalkyl, C₆-C₁₄ aryl, 5-10 membered heteroaryl wherein 1 to 4 ring atoms are independently selected from nitrogen, oxygen or sulfur, 5-10 membered heteroalicyclic wherein 1 to 3 ring atoms are independently nitrogen, oxygen or sulfur, hydroxy, C₁-C₁₀ alkoxy, C₃-C₈ cycloalkoxy, aryloxy, mercapto, alkylthio, arylthio, cyano, halo, carbonyl, thiocarbonyl, O-carbamyl, N-carbamyl, O-thiocarbamyl, N-thiocarbamyl, C-amido, N-amido, C-carboxy, O-carboxy, nitro, silyl, sulfmyl, sulfonyl, amino, and —NR¹⁰R¹¹ where R¹⁰ and R¹¹ are independently selected from the group consisting of hydrogen, C₁-C₄ alkyl, C₃-C₈ cycloalkyl, C₆-C₁₄ aryl, carbonyl, acetyl, sulfonyl, amino, and trifluoromethanesulfonyl, or R¹⁰ and R¹¹, together with the nitrogen atom to which they are attached, combine to form a five-or six-membered heteroalicyclic ring.

A “cycloalkyl” group refers to an all-carbon monocyclic ring (i.e., rings which share an adjacent pair of carbon atoms) of 3 to 8 ring atoms wherein one of more of the rings does not have a completely conjugated pi-electron system e.g., cyclopropyl, cyclobutyl, cyclopentyl, cyclohexyl, cyclobutenyl, cyclopentenyl, cyclohexenyl, and the like. Examples, without limitation, of cycloalkyl groups are cyclopropane, cyclobutane, cyclopentane, cyclopentene, cyclohexane, adamantane, cyclohexadiene, cycloheptane and, cycloheptatriene. A cycloalkyl group may be substituted or unsubstituted. When substituted, the substituent group(s) is one or more, for example one or two groups, individually selected from C₁-C₁₀ alkyl, C₃-C₈ cycloalkyl, C₆-C₁₄ aryl, 5-10 membered heteroaryl wherein 1 to 4 ring atoms are independently selected from nitrogen, oxygen or sulfur, 5-10 membered heteroalicyclic wherein 1 to 3 ring atoms are independently nitrogen, oxygen or sulfur, hydroxy, C₁-C₁₀ alkoxy, C₃-C₈ cycloalkoxy, aryloxy, mercapto, alkylthio, arylthio, cyano, halo, carbonyl, thiocarbonyl, O-carbamyl, N-carbamyl, O-thiocarbamyl, N-thiocarbamyl, C-amido, N-amido, C-carboxy, O-carboxy, nitro, silyl, sulfinyl, sulfonyl, amino, and —NR¹⁰R^{11,} with R¹⁰ and R¹¹ as defined above.

An “alkenyl” group refers to an alkyl group, as defined herein, consisting of at least two carbon atoms and at least one carbon-carbon double bond e.g., ethenyl, propenyl, butenyl or pentenyl and their structural isomeric forms such as 1-or 2-propenyl, 1-, 2-, or 3-butenyl and the like. The alkenyl may comprise 2 to 10 carbon atoms, for example 2 to 8 carbon atoms, 2 to 6 carbon atoms, or 2 to 5 carbon atoms, wherein a numerical range, such as “2 to 10” or “C₂-C₁₀”, refers to each integer in the given range, e.g. “C₂-C₁₀ alkenyl” means that an alkenyl group comprising 2 carbon atoms, 3 carbon atoms, 4 carbon atoms, 5 carbon atoms, 6 carbon atoms, etc., up to and including 10 carbon atoms. An alkenyl or alkene group of this invention may be substituted or unsubstituted. When substituted, the substituent(s) may be selected from the same group disclosed above with regard to alkyl group substitution. Examples of such groups include, but are not limited to, ethenyl, propenyl, butenyl, 1,4-butadienyl, pentenyl, hexenyl, 4-methylhex-1-enyl, 4-ethyl-2-methylhex-1-enyl and the like.

An “alkynyl” group refers to an alkyl group, as defined herein, consisting of at least two carbon atoms and at least one carbon-carbon triple bond e.g., acetylene, ethynyl, propynyl, butynyl, or pentynyl and their structural isomeric forms as described above. The alkynyl group may comprise 2 to 10 carbon atoms, for example 2 to 8 carbon atoms, 2 to 6 carbon atoms, or 2 to 5 carbon atoms, wherein a numerical range, such as “2 to 10” or “C₂-C₁₀”, refers to each integer in the given range, e.g. “C₂-C₁₀ alkynyl” means that an alkynyl group comprising 2 carbon atoms, 3 carbon atoms, 4 carbon atoms, 5 carbon atoms, 6 carbon atoms, etc., up to and including 10 carbon atoms. An alkynyl group of this invention may be substituted or unsubstituted. When substituted, the substituent(s) may be selected from the same group disclosed above with regard to alkyl group substitution. Examples of alkyne groups include, but are not limited to, ethynyl, propynyl, butynyl, and the like.

An “aryl” group refers to an all-carbon monocyclic or fused-ring polycyclic (i.e., rings which share adjacent pairs of carbon atoms) groups of 6 to 14 ring atoms and having a completely conjugated pi-electron system. Examples, without limitation, of aryl groups are phenyl, naphthalenyl and anthracenyl. The aryl group may be substituted or unsubstituted. When substituted, the substituted group(s) is one or more, for example one, two, or three substituents, independently selected from the group consisting of C₁-C₁₀ alkyl, C₃-C₈ cycloalkyl, C₆-C₁₄ aryl, 5-10 membered heteroaryl wherein 1 to 4 ring atoms are independently selected from nitrogen, oxygen or sulfur, 5-10 membered heteroalicyclic wherein 1 to 3 ring atoms are independently nitrogen, oxygen or sulfur, hydroxy, C₁-C₁₀ alkoxy, C₃-C₈ cycloalkoxy, aryloxy, mercapto, alkylthio, arylthio, cyano, halo, trihalomethyl, carbonyl, thiocarbonyl, O-carbamyl, N-carbamyl, O-thiocarbamyl, N-thiocarbamyl, C-amido, N-amido, C-carboxy, O-carboxy, nitro, silyl, sulfinyl, sulfonyl, amino, and —NR¹⁰R¹¹, with R¹⁰ and R¹¹ as defined above. Preferably the substituent(s) is/are independently selected from chloro, fluoro, bromo, methyl, ethyl, hydroxy, methoxy, nitro, carboxy, methoxycarbonyl, sulfonyl, or amino.

A “heteroaryl” group refers to a monocyclic or fused aromatic ring (i.e., rings which share an adjacent pair of atoms) of 5 to 10 ring atoms in which one, two, three or four ring atoms are selected from the group consisting of nitrogen, oxygen and sulfur and the rest being carbon. Examples, without limitation, of heteroaryl groups are pyridyl, pyrrolyl, furyl, thienyl, imidazolyl, oxazolyl, isoxazolyl, thiazolyl, isothiazolyl, pyrazolyl, 1,2,3-triazolyl, 1,2,4-triazolyl, 1,2,3-oxadiazolyl, 1,2,4-oxadiazolyl, 1,2,5-oxadiazolyl, 1,3,4-oxadiazolyl, 1,3,4-triazinyl, 1,2,3-triazinyl, benzofuryl, isobenzofuryl, benzothienyl, benzotriazolyl, isobenzothienyl, indolyl, isoindolyl, 3H-indolyl, benzimidazolyl, benzothiazolyl, benzoxazolyl, quinolizinyl, quinazolinyl, pthalazinyl, quinoxalinyl, cinnnolinyl, napthyridinyl, quinolyl, isoquinolyl, tetrazolyl, 5,6,7,8-tetrahydroquinolyl, 5,6,7,8-tetra-hydroisoquinolyl, purinyl, pteridinyl, pyridinyl, pyrimidinyl, carbazolyl, xanthenyl or benzoquinolyl. The heteroaryl group may be substituted or unsubstituted. When substituted, the substituted group(s) is one or more, for example one or two substituents, independently selected from the group consisting of C₁-C₁₀ alkyl, C₃-C₈ cycloalkyl, C₆-C₁₄ aryl, 5-10 membered heteroaryl wherein 1 to 4 ring atoms are independently selected from nitrogen, oxygen or sulfur, 5-10 membered heteroalicyclic wherein 1 to 3 ring atoms are independently nitrogen, oxygen or sulfur, hydroxy, C₁-C₁₀ alkoxy, C₃-C₈ cycloalkoxy, aryloxy, mercapto, alkylthio, arylthio, cyano, halo, trihalomethyl, carbonyl, thiocarbonyl, O-carbamyl, N-carbamyl, O-thiocarbamyl, N-thiocarbamyl, C-amido, N-amido, C-carboxy, O-carboxy, nitro, silyl, sulfinyl, sulfonyl, amino, and -NR¹⁰R¹¹, with R¹⁰ and R¹¹ as defined above. Preferably the substituent(s) is/are independently selected from chloro, fluoro, bromo, methyl, ethyl, hydroxy, methoxy, nitro, carboxy, methoxycarbonyl, sulfonyl, or amino.

A “heteroalicyclic” group refers to a monocyclic or fused ring of 5 to 10 ring atoms containing one, two, or three heteroatoms in the ring which are selected from the group consisting of nitrogen, oxygen and —S(O)ₙ where n is 0-2, the remaining ring atoms being carbon. The rings may also have one or more double bonds. However, the rings do not have a completely conjugated pi-electron system. Examples, without limitation, of heteroalicyclic groups are pyrrolidine, piperidine, piperazine, morpholine, imidazolidine, tetrahydropyridazine, tetrahydrofuran, thiomorpholine, tetrahydropyridine, and the like. The heteroalicyclic ring may be substituted or unsubstituted. When substituted, the substituted group (s) is one or more, for example one, two, or three substituents, independently selected from the group consisting of C₁-C₁₀ alkyl, C₃-C₈ cycloalkyl, C₆-C₁₄ aryl, 5-10 membered heteroaryl wherein 1 to 4 ring atoms are independently selected from nitrogen, oxygen or sulfur, 5-10 membered heteroalicyclic wherein 1 to 3 ring atoms are independently nitrogen, oxygen or sulfur, hydroxy, C₁-C₁₀ alkoxy, C₃-C₈ cycloalkoxy, aryloxy, mercapto, alkylthio, arylthio, cyano, halo, trihalomethyl, carbonyl, thiocarbonyl, O-carbamyl, N-carbamyl, O-thiocarbamyl, N-thiocarbamyl, C-amido, N-amido, C-carboxy, O-carboxy, nitro, silyl, sulfinyl, sulfonyl, amino, and —NR¹⁰R¹¹, with R¹⁰ and R¹¹ as defined above. The substituent(s) is/are for example independently selected from chloro, fluoro, bromo, methyl, ethyl, hydroxy, methoxy, nitro, carboxy, methoxycarbonyl, sulfonyl, or amino.

A “hydroxyl” group refers to an -OH group.

An “alkoxy” group refers to an —O-unsubstituted alkyl and —O-substituted alkyl group, as defined herein. Examples include and are not limited to methoxy, ethoxy, propoxy, butoxy, and the like.

A “cycloalkoxy” group refers to an —O-cycloalkyl group, as defined herein. One example is cyclopropyloxy.

An “aryloxy” group refers to both an —O-aryl and an —O-heteroaryl group, as defined herein. Examples include and are not limited to phenoxy, napthyloxy, pyridyloxy, furanyloxy, and the like.

A “mercapto” group refers to a —SH group.

An “alkylthio” group refers to both an S-alkyl and an —S-cycloalkyl group, as defined herein. Examples include and are not limited to methylthio, ethylthio, and the like.

An “arylthio” group refers to both an —S-aryl and an —S-heteroaryl group, as defined herein. Examples include and are not limited to phenylthio, napthylthio, pyridylthio, furanylthio, and the like.

A “sulfinyl” group refers to a —S(O)—R″ group, wherein, R″ is selected from the group consisting of hydrogen, hydroxy, alkyl, cycloalkyl, aryl, heteroaryl (bonded through a ring carbon) and heteroalicyclic (bonded through a ring carbon), as defined herein.

A “sulfonyl” group refers to a —S(O)₂R″ group wherein, R″ is selected from the group consisting of hydrogen, hydroxy, alkyl, cycloalkyl, aryl, heteroaryl (bonded through a ring carbon) and heteroalicyclic (bonded through a ring carbon), as defined herein.

A “trihalomethyl” group refers to a —CX₃ group wherein X is a halo group as defined herein e.g., trifluoromethyl, trichloromethyl, tribromomethyl, dichlorofluoromethyl, and the like.

“Carbonyl” refers to a —C(═O)—R″ group, where R″ is selected from the group consisting of hydrogen, alkyl, cycloalkyl, aryl, heteroaryl (bonded through a ring carbon) and heteroalicyclic (bonded through a ring carbon), as defined herein. Representative examples include and the not limited to acetyl, propionyl, benzoyl, formyl, cyclopropylcarbonyl, pyridinylcarbonyl, pyrrolidin-lylcarbonyl, and the like.

A “thiocarbonyl” group refers to a —C(═S)—R″ group, with R″ as defined herein.

“C-carboxy” and “carboxy” which are used interchangeably herein refer to a —C(═O)O—R″ group, with R″ as defined herein, e.g. —COOH, methoxycarbonyl, ethoxycarbonyl, benzyloxycarbonyl, and the like.

An “O-carboxy” group refers to a —OC(═O)R″ group, with R″ as defined herein, e.g. methylcarbonyloxy, phenylcarbonyloxy, benzylcarbonyloxy, and the like.

An “acetyl” group refers to a —C(═O)CH₃ group.

A “carboxylic acid” group refers to a C-carboxy group in which R″ is hydrogen.

A “halo” or “halogen” group refers to fluorine, chlorine, bromine or iodine.

A “cyano” group refers to a —CN group.

A “nitro” group refers to a —NO₂ group.

An “O-carbamyl” group refers to a —OC(═O)NR¹⁰R¹¹ group, with R¹⁰ and R¹¹ as defined herein.

An “N-carbamyl” group refers to a R “OC(═O)NR¹⁰R¹¹ group, with R¹⁰ and R¹¹ as defined herein.

An “O-thiocarbamyl” group refers to a —OC(═S)NR¹⁰R¹¹ group, with R¹⁰ and R¹¹ as defined herein.

An “N-thiocarbamyl” group refers to a R¹¹OC(═S)NR¹⁰— group, with R¹¹ and R^{H} as defined herein.

An “amino” group refers to an —NR¹⁰R¹¹ group, wherein R¹⁰ and R¹¹ are independently hydrogen or unsubstituted lower alkyl, e.g, —NH₂, dimethylamino, diethylamino, ethylarnino, methylamino, and the like.

A “C-amido” group refers to a —C(═O)NR¹⁰R¹¹ group, with R¹⁰ and R¹¹ as defined herein. For example, R¹⁰ is hydrogen or unsubstituted C₁-C₄ alkyl and R¹¹ is hydrogen, C₁-C₄ alkyl optionally substituted with heteroalicyclic, hydroxy, or amino. For example, C(═O)NR¹⁰R¹¹ may be aminocarbonyl, dimethylaminocarbonyl, diethylaminocarbonyl, diethylaminoethylaminocarbonyl, ethylaminoethylaminocarbonyl, and the like.

An “N-amido” group refers to a R¹¹C(═O)NR¹⁰— group, with R¹⁰ and R¹¹ as defined herein, e.g. acetylamino, and the like.

In some embodiments, there is provided a compound of formula Ia:

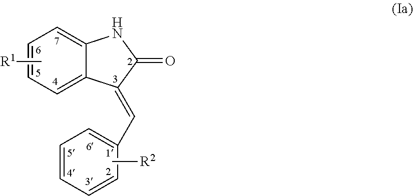

wherein when in and n are both 1,
R¹ is one selected from the group consisting of 6-F; 6-Cl; and 6-OCH₃; and
R² is one selected from the group consisting of 3′OCH₃; 3′-OH; 4′-OCH₃; and 3′CF₃;
and, wherein when m is 1 and n is 2,
R¹ is one selected from the group consisting of 6-F; 6-Cl; and 6-OCH₃; and
R² is one selected from the group consisting of 3′OCH₃; 3′-OH; 4′-OCH₃; and 3′CF₃.

In other embodiments, there is provided a compound of formula IIa:

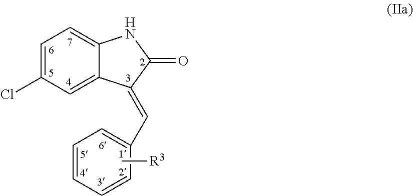

wherein when n is 1,
R³ is one selected from the group consisting of 3′-F, 3′-Cl, 3′-Br, 3′-OCH₃; 3′-OH; 4′-OCH₃, 3′CF₃, 4′-F, 4′-Cl, 4′-Br, 4′-OCH₃; 4′-OH; 3′-OCH₃ and 4′CF₃,
wherein, when n is 2,
R³ is one selected from the group consisting of 3′-F, 3′-Cl, 3′-Br, 3′-OCH₃; 3′-OH; 4′-OCH₃, 3′CF₃, 4′-F, 4′-Cl, 4′-Br, 4′-OCH₃; 4′-OH; 3′-OCH₃ and 4′CF₃.

In certain embodiments, the compound described herein is selected from the group consisting of
6-fluoro-3-(3′-methoxy-benzylidene)-indolin-2-one (also referred to as compound 39 in the following);
6-fluoro-3-(3′-hydroxy-benzylidene)-indolin-2-one;
6-fluoro-3-(4′-methoxy-benzylidene)-indolin-2-one;
6-chloro-3-(3′-methoxy-benzylidene)-indolin-2-one (also referred to as compound 41 in the following);
6-chloro-3-(3′-hydroxy-benzylidene)-indolin-2-one;
6-chloro-3-(4′-methoxy-benzylidene)-indolin-2-one;
6-methoxy-3-(3′-methoxy-benzylidene)-indolin-2-one (also referred to as compound 42 in the following);
6-methoxy-3-(3′-hydroxy-benzylidene)-indolin-2-one;
6-methoxy-3-(4′-methoxy-benzylidene)-indolin-2-one;
6-fluoro-3-(3′-hydroxy-4′-methoxy-benzylidene)-indolin-2-one (also referred to as compound 43 in the following);
6-chloro-3-(3′-hydroxy-4′-methoxy-benzylidene)-indolin-2-one (also referred to as compound 44 in the following);
6-methoxy-3-(3′-hydroxy-4′-methoxy-benzylidene)-indolin-2-one (also referred to as compound 45 in the following);
6-fluoro-3-(3′-trifluoromethyl-benzylidene)-indolin-2-one (also referred to as compound 46 in the following);
6-chloro-3-(3′-trifluoromethyl-benzylidene)-indolin-2-one (also referred to as compound 47 in the following);
6-methoxy-3-(3′-trifluoromethyl-benzylidene)-indolin-2-one (also referred to as compound 48 in the following);
5-chloro-3-(3′-methoxy-benzylidene)-indolin-2-one (also referred to as compound 40 in the following);
5-chloro-3-(3′-hydroxy-benzylidene)-indolin-2-one;
5-chloro-3-(4′-methoxy-benzylidene)-indolin-2-one; and
5-chloro-3-(3′trifluoromethyl-benzylidene)-indolin-2-one.

In other embodiments, the compound of the invention is 6-chloro-3-(3′-trifluoromethyl-benzylidene)-indolin-2-one.

The compounds of the inventions or pharmaceutically acceptable salts or prodrugs thereof, can be synthesized using techniques commonly known in the art and readily available starting materials. The compounds of the invention may also be synthesized according to known techniques such as the synthesis methods described in U.S. Pat. No. 6,469,032 issued to Tang et al the content of which is incorporated in its entirety by reference herein. Accordingly, the compounds of the invention can be prepared by reacting the proper 2-indolinone compound with an appropriate aldehyde according to method A or B as described in examples 5.1 and 5.4 of U.S. Pat. No. 6,469,032.

Compounds of the invention or pharmaceutically acceptable salts or prodrugs thereof can also be obtained in the synthesis scheme as described in Example 1 Scheme 1. In more detail, the compounds of formula I or II can be prepared by reacting an oxindole of formula (III)

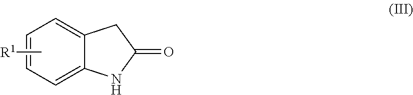

with an aldehyde of formula (IV)

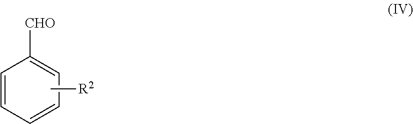

in the presence of a base.

In this context, the R¹ and R² substituents in the respective formula III and IV are defined in the compounds of formula I or II described herein. In some embodiments, the base can include an amine base. The amine base includes but is not limited to cyclic amines such as aziridine, piperidine and N-methylpiperidine. In some embodiments, the base can be piperidine.

In this context, the compounds of formula I or II as described herein may be present as E-isomers, Z-isomers, or a mixture of both E and Z isomers.

The compounds according to the invention can be administered in pharmaceutical formulations or compositions, either alone or in combination with other pharmacologically active compounds.

Examples of other active ingredients that may be included in a pharmaceutical composition include, but are not limited to, a nucleic acid alkylator, a nucleoside analogue, an anthracycline, an antibiotic, an aromatase inhibitor, a folate antagonist, an estrogen receptor modulator, an inorganic aresenate, a microtubule inhibitor, a nitrosourea, an osteoclast inhibitor, a platinum containing compound, a retinoid, a topoisomerase 1 inhibitor, a topoisomerase 2 inhibitor, a thymidylate synthase inhibitor, an aromatase inhibitor, a cyclo-oxygenase inhibitor, an isoflavone, a tyrosine kinase inhibitor, a growth factor, a bisphosphonate, and a monoclonal antibody.

Alkylators that may be included in the pharmaceutical composition of the present invention include but are not limited to busulfan (Myleran®, Busilvex®), chlorambucil (Leukeran®), ifosfamide (Mitoxana®, with or without MESNA), cyclophosphamide (Cytoxan®, Neosar®), glufosfamide, melphalan/L-PAM (Alkeran®), dacarbazine (DTIC-Dome®), and temozolamide (Temodar®). As an illustrative example, the compound 2-bis[(2-chloroethyl)amino]tetra-hydro-2H-1,3,2-oxazaphosphorine, 2-oxide, also commonly known as cyclophosphamide, is an alkylator used in the treatment of stages III and IV malignant lymphomas, multiple myeloma, leukemia, mycosis fungoides, neuroblastoma, ovarian adenocarcinoma, retinoblastoma, and carcinoma of the breast.

Nucleoside analogues that may be included in the pharmaceutical composition of the present invention include, but are not limited to, cytarabine (Cytosar®) and gemcitabine (Gemzar®), two fluorinated deoxycytidine analogues, fludarabine (Fludara®), a purine analog, 6-mercaptopurine (Puri-Nethol®) and its prodrug azathioprine (Imuran®).

Anthracyclines that may be included in the pharmaceutical composition of the present invention include, but are not limited to, doxorubicin (Adriamycin®, Doxil®, Rubex®), mitoxantrone (Novantrone®), idarubicin (Idamycin®), valrubicin (Valstar®), and epirubicin (Ellence®). As one example, the compound (8S,1S)-10-(4-amino-5-hydroxy-6-methyl-tetrahydro-2H-pyran-2-yloxy)-6,8,11-trihydroxy-8-(2-hydroxyacetyl)-1-methoxy-7,8,9,10-tetrahydrotetracene-5,12-dione, more commonly known as doxorubicin, is a cytotoxic anthracycline antibiotic isolated from cultures of Streptomyces peucetius var. caesius. Doxorubicin has been used successfully to produce regression in disseminated neoplastic conditions such as acute lymphoblastic leukemia, acute myeloblastic leukemia, Wilm's tumour, neuroblastoma, soft tissue and bone sarcomas, breast carcinoma, ovarian carcinoma, transitional cell bladder carcinoma, thyroid carcinoma, lymphomas of both Hodgkin and non-Hodgkin types, bronchogenic carcinoma, and gastric carcinoma.

Antibiotics that may be included in the pharmaceutical composition of the present invention include but are not limited to dactinomycin, actinomycin D (Cosmegen®), daunorubicin/daunomycin (Cerubidine®, DanuoXome®), bleomycin (Blenoxane®), epirubicin (Pharmorubicin®) and mitoxantrone (Novantrone®). Aromatase inhibitors useful in the practice of the present invention include but are not limited to anastrozole (Arimidex®) and letroazole (Femara®). Bisphosphonate inhibitors that may be included in the pharmaceutical composition of the present invention include but are not limited to zoledronate (Zometa®).

Cyclooxygenase inhibitors that may be included in the pharmaceutical composition of the present invention include but are not limited to acetylsalicylic acid (Aspirin®), celecoxib (Celebrex®) and rofecoxib (Vioxx®, Ceoxx®, Ceeoxx®). Estrogen receptor modulators that may be included in the composition of the present invention include but are not limited to tamoxifen (Nolvadex®) and fulvestrant (Faslodex®). Folate antagonists that may be included in the composition of the present invention include but are not limited to methotrexate (Trexall®, Rheumatrex®) and trimetrexate (Neutrexin®). As an illustrative example, the compound (S)-2-(4-((2,4-diaminopteridin-6-yl)methyl)methylamino)benzamido)pentanedioic acid, commonly known as methotrexate, is an antifolate drug that has been used in the treatment of gestational choriocarcinoma and in the treatment of patients with chorioadenoma destruens and hydatiform mole. It is also useful in the treatment of advanced stages of malignant lymphoma and in the treatment of advanced cases of mycosis fungoides.

Inorganic arsenates that may be included in the pharmaceutical composition of the present invention include but are not limited to arsenic trioxide (Trisenox®). Microtubule inhibitors (as used herein, a “microtubule inhibitor” is any agent that interferes with the assembly or disassembly of microtubules) that may be included in the composition of the present invention include but are not limited to vincristine (Oncovin®), vinblastine (Velban®), paclitaxel (Taxol®, Paxene®), vinorelbine (Navelbine®), docetaxel (Taxotere®), epothilone B or D or a derivative of either, and discodermolide or its derivatives.

Nitrosoureas that may be included in the pharmaceutical composition of the present invention include but are not limited to procarbazine (Matulane®), lomustine (CeeNU®), carmustine (BCNU®, BiCNU®, Gliadel Wafer®), and estramustine (Emcyt®). Nucleoside analogs that may be included in the pharmaceutical composition of the present invention include but are not limited to 6-mercaptopurine (Purinethol®), 5-fluorouracil (Adrucil®), 6-thioguanine (Thioguanine®), hydroxyurea (Hydrea®), cytarabine (Cytosar-U®, DepoCyt®), floxuridine (FUDR®), fludarabine (Fludara®), pentostatin (Nipent®), cladribine (Leustatin®, 2-CdA®), gemcitabine (Gemzar®), and capecitabine (Xeloda®). As an illustrative example, the compound 5-fluoro-2,4(1H,3H)-pyrimidinedione, also commonly known as 5-fluorouracil, is an antimetabolite nucleoside analogue effective in the palliative management of carcinoma of the colon, rectum, breast, stomach, and pancreas in patients who are considered incurable by surgical or other means. Another example of a nucleoside analogue is Gemcitabine. Gemcitabine is 2′-deoxy-2′,2′-difluoro-cytidine. It is commercially available as the monohydrochloride salt, and as the beta-isomer. It is also known chemically as 1-(4-amino-2-oxo-1-H-pyrimidin-1-yl)-2-desoxy-2,2-difluororibose.

An illustrative example of an osteoclast inhibitor that may be included in the pharmaceutical composition of the present invention is pamidronate (Aredia®). Platinum compounds that may be included in the pharmaceutical composition of the present invention include, but are not limited to, cisplatin (Platinol®) and carboplatin (Paraplatin®). Retinoids that may be included in the pharmaceutical composition of the present invention include but are not limited to tretinoin, ATRA (Vesanoid®), alitretinoin (Panretin®), and bexarotene (Targretin®). Topoisomerase 1 inhibitors that may be included in the pharmaceutical composition of the present invention include, but are not limited to, topotecan (Hycamtin®) and irinotecan (Camptostar®, Camptothecan-11®). Topoisomerase 2 inhibitors that may be included in the pharmaceutical composition of the present invention include, but are not limited to, etoposide (Etopophos®, Vepesid®) and teniposide (Vumon®).

Examples of other suitable tyrosine kinase inhibitors that may be included in the pharmaceutical composition of the present invention include, but are not limited to, dasatinib (Sprycel®), erlotinib (Tarceva®), gefitinib (Iressa®), imatinib (Gleevec®), lapatinib (Tykerb®), sorafenib (Nexavar®) and vandetanib (Zactima®). Examples of a (recombinant) growth factor that may be included in the pharmaceutical composition of the present invention include, but are not limited to, interleukin-11, interferon-α-2b and interleukin-2. An illustrative example of a thymidylate synthase inhinitor that may be included in the pharmaceutical composition of the present invention is Raltitrexed®. Examples of a monoclonal antibody that may be included in the pharmaceutical composition of the present invention include, but are not limited to, rituximab (MabThera®) or cetuximab (Erbitux®).

In some embodiments, there is provided a pharmaceutical composition. The pharmaceutical composition can include a compound according to the invention or salt or prodrug thereof and a pharmaceutically acceptable carrier or excipient.

A “pharmaceutical composition” refers to a mixture of one or more of the compounds described herein, or physiologically/pharmaceutically acceptable salts or prodrugs thereof, with other chemical components, such as physiologically/pharmaceutically acceptable carriers and excipients. The purpose of a pharmaceutical composition is to facilitate administration of a compound to an organism.

The compounds of formula I or II may also act as a prodrug. A “prodrug” refers to an agent which is converted into the parent drug in vivo. Prodrugs are often useful because, in some situations, they may be easier to administer than the parent drug. They may, for instance, be bioavailable by oral administration whereas the parent drug is not. The prodrug may also have improved solubility in pharmaceutical compositions over the parent drug. An example, without limitation, of a prodrug would be a compound of the present invention which is administered as an ester (the “prodrug”) to facilitate transmittal across a cell membrane where water solubility is detrimental to mobility but then is metabolically hydrolyzed to the carboxylic acid, the active entity, once inside the cell where water solubility is beneficial. A prodrug may be converted into the parent drug by various mechanisms, including enzymatic processes and metabolic hydrolysis.

A further example of a prodrug might be a short polypeptide, for example, without limitation, a 2-10 amino acid polypeptide, bonded through a terminal amino group to a carboxy group of a compound of this invention wherein the polypeptide is hydrolyzed or metabolized in vivo to release the active molecule. The prodrugs of compounds of Formula I or II are within the scope of this invention.

Additionally, it is contemplated that compounds of Formula I or II would be metabolized by enzymes in the body of the organism such as a human being to generate a metabolite that can modulate the activity of a protein tyrosine kinase. Such metabolites are within the scope of the present invention.

As used herein, a “physiologically/pharmaceutically acceptable carrier” refers to a carrier or diluent that does not cause significant irritation to an organism and does not abrogate the biological activity and properties of the administered compound.

A “pharmaceutically acceptable excipient” refers to an inert substance added to a pharmaceutical composition to further facilitate administration of a compound. Examples, without limitation, of excipients include calcium carbonate, calcium phosphate, various sugars and types of starch, cellulose derivatives, gelatine, vegetable oils and polyethylene glycols.

As used herein, the term “pharmaceutically acceptable salt” refers to those salts which retain the biological effectiveness and properties of the parent compound. Such salts include, but are not restricted to: (1) an acid addition salt which is obtained by reaction of the free base of the parent compound with inorganic acids such as hydrochloric acid, hydrobromic acid, nitric acid, phosphoric acid, sulfuric acid, and perchloric acid and the like, or with organic acids such as acetic acid, oxalic acid, (D) or (L) malic acid, maleic acid, methanesulfonic acid, ethanesulfonic acid, p-toluenesulfonic acid, salicylic acid, tartaric acid, citric acid, succinic acid or malonic acid and the like, preferably hydrochloric acid or (L)-malic acid; or (2) salts formed when an acidic proton present in the parent compound either is replaced by a metal ion, e.g., an alkali metal ion, such as sodium or potassium, an alkaline earth ion, such as magnesium or calcium, or an aluminium ion; or coordinates with an organic base such as ethanolamine, diethanolamine, triethanolamine, tromethamine, N-methylglucamine, and the like.

The terms “protein tyrosine kinase related disease or disorder” is used herein to refer to a condition involving protein tyrosine kinase activity, including aberrant protein tyrosine kinase activity. Examples for such diseases and disorders are aberrant cell proliferative diseases, such as cancers, fibrotic and mesangial disorders, abnormal angiogenesis and vasculogenesis, wound healing, psoriasis, diabetes mellitus, and inflammation; aberrant differentiation conditions which include but are not limited to neurodegenerative disorders, slow wound healing rates and tissue grafting techniques; and aberrant cell survival conditions. Aberrant cell survival conditions relate to conditions in which programmed cell death (apoptosis) pathways are activated or abrogated. A number of protein kinases are associated with the apoptosis pathways. Aberrations in the function of any one of the protein kinases could lead to cell immortality or premature cell death. In some embodiments, the protein tyrosine kinase related disorders may include RTK-related disorders, for example an IGF-1R related disorder, an EphA2 related disorder or a Tyro3 related disorder. These disorders may be hepatocellular carcinoma, breast cancer, colon cancer and lung cancer.

The compounds of the present invention bind with high specificity and selectivity to protein tyrosine kinases, especially receptor tyrosine kinases (RTKs). Exemplary RTKs that may be bound by the compounds of the present invention are, without limitation, EGFR, HER2, HER3, HER4, IR, IGF-1R, IRR, PDGFR CSFIR, C-Kit, C-fms, Flk-1R, Flk4, KDR/Flk1, Flt-1, FGFR-1R, FGFR-2R, FGFR-3R, FGFR-4R, EphA1, EphA2, EphA3, EphA4, EphA5, EphA6, EphA7, EphA8, EphA10 and Tyro3. Upon binding, the compounds of the present invention reduce or abrogate protein tyrosine kinase-mediated cellular signaling. Without being bound to any particular theory, it is believed that the compounds of the invention will minimize and obliterate solid tumors by specifically inhibiting the activity of the tyrosine kinases, or will at least modulate or inhibit tumor growth and/or metastases. A precise understanding of the mechanism by which the compounds of the invention inhibit protein tyrosine kinase signalling is not required in order to practice the present invention. However, while not hereby bound to any particular mechanism or theory, it is believed that the compounds interact with amino acids of the protein tyrosine kinase in the ATP binding region or in close proximity thereto, through non-covalent interactions such as hydrogen bonding, Van de Waals interactions and ionic bonding. Therefore, this blocks the binding of ATP and thus the phosphorylation of other proteins. In this context, the specificity of the compounds of the present invention for a particular protein tyrosine kinase may be conferred by interactions between the constituents around the oxindole core of the compounds of the invention with the amino acid domains specific to individual protein tyrosine kinases. Thus, different indolinone substituents may contribute to preferential binding to particular protein tyrosine kinases.

In some embodiments, the invention relates to a method for modulation of the catalytic activity of a protein kinase such as a protein tyrosine kinase described above. The method includes contacting said protein kinase with the compound according to the invention, salt or prodrug thereof.

As used herein, the term “modulate” refers to a change in the activity of a protein kinase as described herein. For example, modulation may cause an increase or a decrease in protein activity, binding characteristics, or any other biological, functional or immunological properties of the tyrosine kinase.

The compounds of the present invention are useful as inhibitors of protein tyrosine kinase-mediated cellular signalling. Because protein tyrosine kinases play a critical role in, inter alia, cellular proliferation, apoptosis, differentiation and migration, the compounds of the invention have anti-proliferative activity and thus can be utilized in the treatment of diseases and disorders that are connected to an inappropriate or abnormal protein tyrosine kinase function, in particular increased protein tyrosine kinase activity, or that involve protein tyrosine kinase function. Diseases and disorders that may thus be treated and/or prevented by the compounds of the present invention are by way of example, without being limited to these diseases and disorders, hyperproliferative disorders and cancers such as RTK-related disorders, for example an IGF-1R related disorder, an EphA2 related disorder or a Tyro3 related disorder. These disorders may be hepatocellular carcinoma, breast cancer, colon cancer and lung cancer.

Therefore, in some embodiments, the present invention is directed to a method for the treatment or prevention of protein tyrosine kinase-related disease or disorder in an organism. The method includes the administration of a pharmaceutically active amount of a compound according to the invention to a subject in need thereof. The subject may be a mammal. A mammal may include but is not limited to organisms such as mice, rats, rabbits, guinea pigs, monkeys and apes and humans.

“Treat”, “treating” and “treatment” refer to a method of alleviating or abrogating a protein tyrosine kinase related disease or disorder and/or its attendant symptoms.

“Prevent”, “preventing” and “prevention” refer to a method of hindering a protein tyrosine kinase related disease or disorder from occurring, i.e. a prophylactic method.

“Organism” refers to any living entity comprised of at least one cell. A living organism can be as simple as, for example, a single eukaryotic cell or as complex as a mammal, including a human being.

In this context, it should be noted that for the efficacy of a compound of the invention as a pharmaceutical, a number of variables besides binding affinity may play a crucial role. Accordingly, compounds of the invention can be specifically selected for use as a pharmaceutical not only based on the determined binding specificity and affinity, but also based on other factors, such as bioavailability, severity of side effects caused, metabolic conversion of the compound, half-life of the compound in the organism and the like.

A compound of the present invention or a pharmaceutically acceptable salt or prodrug thereof, can be administered as such to a human patient or can be administered in pharmaceutical compositions in which the foregoing materials are mixed with suitable carriers or excipient(s). Techniques for formulation and administration of drugs may be found in “Remington's Pharmacological Sciences,” Mack Publishing Co., Easton, Pa., latest edition.

As used herein, “administer” or “administration” refers to the delivery of a compound of Formula (I) or (II) or a pharmaceutically acceptable salt or prodrug thereof or of a pharmaceutical composition containing a compound of Formula (I) or (II) or a pharmaceutically acceptable salt or prodrug thereof of this invention to an organism for the purpose of prevention or treatment of a protein tyrosine kinase related disease or disorder.

Suitable routes of administration may include, without limitation, oral, rectal, transmucosal or intestinal administration or intramuscular, subcutaneous, intramedullary, intrathecal, direct intraventricular, intravenous, intravitreal, intraperitoneal, intranasal, or intraocular injections. In some embodiments, the routes of administration are oral and parenteral.

Alternatively, one may administer the compound in a local rather than systemic manner, for example, via injection of the compound directly into a vessel, optionally in a depot or sustained release formulation.

Pharmaceutical compositions of the present invention may be manufactured by processes well known in the art, e.g., by means of conventional mixing, dissolving, granulating, drageemaking, levigating, emulsifying, encapsulating, entrapping or lyophilizing processes.

Pharmaceutical compositions for use in accordance with the present invention may be formulated in conventional manner using one or more physiologically acceptable carriers comprising excipients and auxiliaries which facilitate processing of the active compounds into preparations which can be used pharmaceutically. Proper formulation is dependent upon the route of administration chosen.

For injection, the compounds of the invention may be formulated in aqueous solutions, preferably in physiologically compatible buffers such as Hanks' solution, Ringer's solution, or physiological saline buffer. For transmucosal administration, penetrants appropriate to the barrier to be permeated are used in the formulation. Such penetrants are generally known in the art.

For oral administration, the compounds can be formulated by combining the active compounds with pharmaceutically acceptable carriers well known in the art. Such carriers enable the compounds of the invention to be formulated as tablets, pills, lozenges, dragees, capsules, liquids, gels, syrups, slurries, suspensions and the like, for oral ingestion by a patient. Pharmaceutical preparations for oral use can be made using a solid excipient, optionally grinding the resulting mixture, and processing the mixture of granules, after adding other suitable auxiliaries if desired, to obtain tablets or dragee cores. Useful excipients are, in particular, fillers such as sugars, including lactose, sucrose, mannitol, or sorbitol, cellulose preparations such as, for example, maize starch, wheat starch, rice starch and potato starch and other materials such as gelatine, gum tragacanth, methyl cellulose, hydroxypropylmethyl-cellulose, sodium carboxymethylcellulose, and/or polyvinyl-pyrrolidone (PVP). If desired, disintegrating agents may be added, such as cross-linked polyvinyl pyrrolidone, agar, or alginic acid. A salt such as sodium alginate may also be used.

Dragee cores are provided with suitable coatings. For this purpose, concentrated sugar solutions may be used which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical compositions which can be used orally include push-fit capsules made of gelatine, as well as soft, sealed capsules made of gelatine and a plasticizer, such as glycerol or sorbitol. The push-fit capsules can contain the active ingredients in admixture with a filler such as lactose, a binder such as starch, and/or a lubricant such as talc or magnesium stearate and, optionally, stabilizers. In soft capsules, the active compounds may be dissolved or suspended in suitable liquids, such as fatty oils, liquid paraffin, or liquid polyethylene glycols. Stabilizers may be added in these formulations, also.

The compounds may also be formulated for parenteral administration, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulating materials such as suspending, stabilizing and/or dispersing agents.

Pharmaceutical compositions for parenteral administration include aqueous solutions of a water soluble form, such as, without limitation, a salt, of the active compound.

Additionally, suspensions of the active compounds may be prepared in a lipophilic vehicle. Suitable lipophilic vehicles include fatty oils such as sesame oil, synthetic fatty acid esters such as ethyl oleate and triglycerides, or materials such as liposomes. Aqueous injection suspensions may contain substances which increase the viscosity of the suspension, such as sodium carboxymethyl cellulose, sorbitol, or dextran. Optionally, the suspension may also contain suitable stabilizers and/or agents that increase the solubility of the compounds to allow for the preparation of highly concentrated solutions.

Alternatively, the active ingredient may be in powder form for constitution with a suitable vehicle, e.g., sterile, pyrogen-free water, before use.

The compounds may also be formulated in rectal compositions such as suppositories or retention enemas, using, e.g., conventional suppository bases such as cocoa butter or other glycerides.

In addition to the formulations described previously, the compounds may also be formulated as depot preparations. Such long acting formulations may be administered by implantation (for example, subcutaneously or intramuscularly) or by intramuscular injection. A compound of this invention may be formulated for this route of administration with suitable polymeric or hydrophobic materials (for instance, in an emulsion with a pharmacologically acceptable oil), with ion exchange resins, or as a sparingly soluble derivative such as, without limitation, a sparingly soluble salt.

A non-limiting example of a pharmaceutical carrier for the hydrophobic compounds of the invention is a cosolvent system comprising benzyl alcohol, a nonpolar surfactant, a water-miscible organic polymer and an aqueous phase such as the VPD co-solvent system. VPD is a solution of 3% w/v benzyl alcohol,8% w/v of the nonpolar surfactant Polysorbate 80, and 65% w/v polyethylene glycol 300, made up to volume in absolute ethanol. The VPD co-solvent system (VPD: D5W) consists of VPD diluted 1:1 with a 5% dextrose in water solution. This cosolvent system dissolves hydrophobic compounds well, and itself produces low toxicity upon systemic administration.

Naturally, the proportions of such a co-solvent system may be varied considerably without destroying its solubility and toxicity characteristics. Furthermore, the identity of the co-solvent components may be varied: for example, other lowtoxicity nonpolar surfactants may be used instead of Polysorbate 80, the fraction size of polyethylene glycol may be varied, other biocompatible polymers may replace polyethylene glycol, e.g., polyvinyl pyrrolidone, and other sugars or polysaccharides may substitute for dextrose.

Alternatively, other delivery systems for hydrophobic pharmaceutical compounds may be employed. Liposomes and emulsions are well known examples of delivery vehicles or carriers for hydrophobic drugs. In addition, certain organic solvents such as dimethylsulfoxide also may be employed, although often at the cost of greater toxicity.

Additionally, the compounds may be delivered using a sustained-release system, such as semi permeable matrices of solid hydrophobic polymers containing the therapeutic agent.

Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for stabilization may be employed.

The pharmaceutical compositions herein also may comprise suitable solid or gel phase carriers or excipients. Examples of such carriers or excipients include, but are not limited to, calcium carbonate, calcium phosphate, various sugars, starch, cellulose derivatives, gelatine, and polymers such as polyethylene glycols.

Many of the compounds of the invention may be provided as physiologically acceptable salts wherein the claimed compound may form the negatively or the positively charged species. Examples of salts in which the compound forms the positively charged moiety include, without limitation, the sodium, potassium, calcium and magnesium salts formed by the reaction of a carboxylic acid or sulfonic acid group in the compound with an appropriate base (e.g. sodium hydroxide (NaOH), potassium hydroxide (KOH), Calcium hydroxide (Ca(OH)₂), etc.).

Pharmaceutical compositions suitable for use in the present invention include compositions wherein the active ingredients are contained in an amount sufficient to achieve the intended purpose, e.g., the inhibition of protein tyrosine kinase function or the treatment or prevention of a protein tyrosine kinase related disease or disorder.

More specifically, a therapeutically effective amount means an amount of compound effective to prevent, alleviate or ameliorate symptoms of disease or prolong the survival of the subject being treated. Determination of a therapeutically effective amount is well within the capability of those skilled in the art, especially in light of the detailed disclosure provided herein.

For any compound used in the methods of the invention, the therapeutically effective amount or dose can be estimated initially from cell culture assays. Then, the dosage can be formulated for use in animal models so as to achieve a circulating concentration range that includes the IC₅₀ as determined in cell culture (i.e., the concentration of the test compound which achieves a half-maximal inhibition of the protein tyrosine kinase activity). Such information can then be used to more accurately determine useful doses in humans.

Toxicity and therapeutic efficacy of the compounds described herein can be determined by standard pharmaceutical procedures in cell cultures or experimental animals, e.g., by determining the IC₅₀ and the ID₅₀ for a subject compound. The data obtained from these cell culture assays and animal studies can be used in formulating a range of dosage for use in humans. The dosage may vary depending upon the dosage form employed and the route of administration utilized. The exact formulation, route of administration and dosage can be chosen by the individual physician in view of the patient's condition.

Dosage amount and interval may be adjusted individually to provide plasma levels of the active species which are sufficient to maintain the protein tyrosine kinase inhibiting effects. These plasma levels are referred to as minimal effective concentrations (MECs). The MEC will vary for each compound but can be estimated from in vitro data, e.g., the concentration necessary to achieve 50-90% inhibition of a certain protein tyrosine kinase.

Dosages necessary to achieve the MEC will depend on individual characteristics and route of administration. HPLC assays or bioassays can be used to determine plasma concentrations.

Dosage intervals can also be determined using MEC value. In this context, compounds should be administered using a regimen that maintains plasma levels above the MEC for 10-90% of the time, preferably between 30-90% and most preferably between 50-90%.

In cases of local administration or selective uptake, the effective local concentration of the drug may not be related to plasma concentration and other procedures known in the art may be employed to determine the correct dosage amount and interval.

The amount of a composition administered will, of course, be dependent on the subject being treated, the severity of the affliction, the manner of administration, the judgment of the prescribing physician, etc.

The compositions may, if desired, be presented in a pack or dispenser device, such as a kit approved by a regulatory authority, such as EMEA or FDA, which may contain one or more unit dosage forms containing the active ingredient. The pack may for example comprise metal or plastic foil, such as a blister pack. The pack or dispenser device may be accompanied by instructions for administration.

The pack or dispenser may also be accompanied by a notice associated with the container in a form prescribed by a governmental agency regulating the manufacture, use or sale of pharmaceuticals, which notice is reflective of approval by the agency of the form of the compositions or of human or veterinary administration. Compositions comprising a compound of the invention formulated in a compatible pharmaceutical carrier may also be prepared, placed in an appropriate container, and labeled for treatment of an indicated condition.

It is also an aspect of this invention that a compound described herein, or its salt or prodrug thereof, might be combined with other agents for the treatment of the diseases and disorders discussed above.

The present invention also encompasses a method of identifying a protein kinase inhibitor having specific efficacy against hepatocellular carcinoma. This method is particularly useful to identify a protein kinase inhibitor against hepatocellular carcinoma (HCC) and includes: i) incubating a candidate compound with a hepatocellular carcinoma cell line; ii) determining cell viability; and iii) comparing the determined cell viability with the candidate compound's effects on a normal liver cell line to identify a compound with specific activity against hepatocellular carcinoma cells.

In this context, it is noted that the term “specific activity against hepatocellular carcinoma” means “reduced toxicity against normal liver cells” and does not mean that the compounds identified by the screening method have reduced or no activity against other cancer type/ protein tyrosine kinase related disease or disorder. Rather, compounds identified by the screening method of the invention can also have activity against other cells (in vitro and in vivo) that are affected by a protein tyrosine kinase related disease or disorder.

In one embodiment, the hepatocellular carcinoma (HCC) cell line used for screening can be any HCC cell lines which are known to persons skilled in the art. These cell lines may include but are not limited to HepG2, HuH7, SNU398, SNU368, Hep3B, Hepa1c1c7, LH86, SK-Hep-1, PLC/PRF/5, Hs817.T and MHCC97 cell lines.

In another embodiment, the normal liver cell line used for screening is THLE2.

In one embodiment, the method is a phenotype-based screening assay.

In order that the invention may be readily understood and put into practical effect, particular embodiments will now be described by way of the following non-limiting examples.

### EXAMPLES

The following experimental examples are provided to further illustrate the present invention and are not intended to be limiting to the scope of the invention.

### Chemicals

The compounds according to the invention described herein (Compounds 39-48; see Table 1 infra) were synthesized using methods as described in U.S. provisional application “Functionalized Indolinones” serial number U.S. 61/105,206 the content of which is incorporated by reference herein in its entirety for all purposes. Sunitinib was obtained from LC Laboratories (Boston, Mass.). All other reagents were obtained from Sigma-Aldrich (St. Louis, Mo.) unless otherwise stated.

### Cell Culture

HuH7 was obtained from Dr. P. Hofschneider (Max Planck Institute, Martinsried, Germany). HepG2, SK-Hep1, Hep3B, PLC/PRF/5, THLE2 and Hs817.T cells were from ATCC (Manassas, Va.). HepG2, Hep3B and PLC/PRF/5 were maintained in MEM, THLE2 in BEGM (Lonza, Basel, Switzerland), all other cells in DMEM. All cell cultures were supplemented with ATCC recommended reagents obtained from Invitrogen (Carlsbad, Calif.).

### Quantitative Real-Time PCR

Total RNA was harvested with Trizol (Invitrogen) as previously described (14) and purified with RNeasy column (Qiagen, Valencia, Calif.). Two μg RNA was used for cDNA synthesis using SuperScript III (Invitrogen), performed according to manufacturer's instruction. Quantitative real-time-PCR was performed using Applied Biosystems 7300 (ABI, Foster City, Calif.) for AFP (NM_{—}001134) with 18S mRNA as control. Samples were prepared in triplicates with 4 μL of prediluted cDNA each. The primer sequences were: AGCTTGGTGGTGGATGAAAC (AFP forward; SEQ ID NO:1); TCTTGCTTCATCGTTTGCAG (AFP reverse; SEQ ID NO:2); CGGCTTAATTTGACTCAACACG (18S forward; SEQ ID NO:3); TTAGCATGCCAGAGTCTCGTTC (18S reverse; SEQ ID NO:4). Data were obtained as average C_{T} values, and normalized against control as AC_{T}. Expression changes in AFP transcripts between normal vs. tumor tissue were expressed as fold change using 2^{ΔΔCT (difference between the ΔC}_{T} of the matched pairs).

### Immunoblot Assay

Protein concentrations were assayed by BCA method (Pierce, Rockford, Ill.). Samples were resolved using 6-10% SDS-PAGE and transferred to nitrocellulose or PVDF membranes by tank transfer. Immunodetection was by chemiluminescence (SuperSignal, Pierce) using specific antibodies diluted in PBS with 0.05% (v/v) Tween 20 and 5% (w/v) powdered milk or BSA. Anti-phospho-IGF-1R, anti-phospho-EphA2, anti-phospho-Erk, anti-phospho-Akt, anti-Bax, anti-BCL-xL were from Cell Signaling Technology (Beverly, Mass.); anti-PCNA and anti-HSP60 from Santa Cruz Biotechnology (Santa Cruz, Calif.); anti-cyclin-D1 from BD Pharmingen (San Jose, Calif.). Secondary antibodies were anti-mouse and anti-rabbit HRP-conjugated antibodies (Pierce).

### Immunoprecipitation

HuH7 cells were treated as described previously for immunoblot assays and harvested with lysis buffer (1% NP-40, 20 mM Tris-HCl, 137 mM NaCl, 10% glycerol, 2 mM EDTA, 1 mM sodium orthovanadate, 10 μg/mL aprotinin, 10 mg/μL leupeptin). Lysates (300 μg) was diluted in 500 μL of HNTG buffer (250 mM HEPES, 150 mM NaCl, 10% glycerol, 0.1% Triton-X) and incubated with 30 μL of Protein A/G mix (GE Healthcare, Waukesha, Wis.) and 2 μg of anti-EGFR (Upstate, Lake Placid, N.Y.), anti-EphA2 (Santa Cruz) or anti-Tyro3 (Bethyl Laboratories, Montgomery, Tex.)) overnight at 4° C. Resulting beads were washed with HNTG buffer before boiling in 20 μL SDS-PAGE sample buffer for electrophoresis. Subsequent immunodetection was with anti-phosphotyrosine (4G10, Upstate) and loading determined by stripping and re-probing with respective primary antibodies.

### Cell Viability Assay

Cells were plated at 5000-7000 cells/per well and incubated for 24 h before treatment. Various concentrations (0-10 μM) of test compounds (Compound 39-48 and sunitinib) were added to cells for 72 h with 8 replicates. Cell Titer-Glo (Promega, Madison, Wis.) assay was performed according to manufacturer's instruction with luminescence detected on SpectraMax M5 (Molecular Devices, Sunnyvale, Calif.). Data was expressed as percentage of viability vs. vehicle-treated controls.

### Caspase-3 Activity Assay

Cells were subjected to Compound 47 treatment for 24 h prior to harvesting. Caspase-3 activities were determined by as previously described (Ho HK, Pok S, Streit S, Ruhe J E, Hart S, Lim K S, et al. Fibroblast growth factor receptor 4 regulates proliferation, anti-apoptosis and alpha-fetoprotein secretion during hepatocellular carcinoma progression and represents a potential target for therapeutic intervention. J Hepatol 2009 January; 50(1):118-127). Data were expressed as RFU/μg lysate/h incubation and error bars in terms of SD with n=3.

### Phospho-RTK profiling

Simultaneous determination of multiple RTK phosphorylation was achieved with Human Phospho-RTK array (RnD Systems, Minneapolis, Minn.). HuH7 cells were subjected to vehicle, Compound 47 or sunitinib treatments for 24 h under serum-free conditions. Cells harvesting, hybridization with RTK array and incubation with anti-phosphotyrosine were performed according to manufacturer's instruction. Imaging and quantification of the spots intensities was with Fujifilm LAS-3000 (Tokyo, Japan). Average signal of the duplicate spots for each RTK was determined.

### Example 1

### Synthesis of Compounds 39-48, Table 1

Scheme 1: Synthetic Pathway of Compounds 39-48 According to the Present Invention

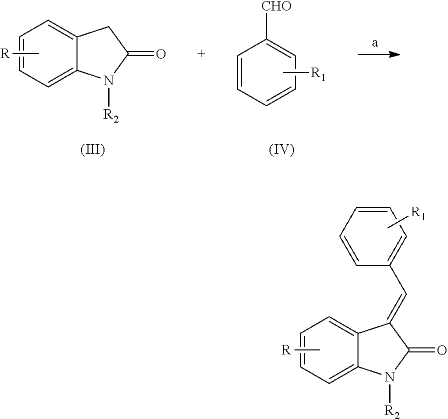

The compounds 39-48 according to the present invention were synthesized by a Knoevenagel reaction between the aldehyde of formula IV and oxindole of formula III. The condensation was carried out in ethanol with piperidine as base catalyst by reflux or in a microwave reactor (Scheme 1).

TABLE 1
Utilized Indolinones

| Compound | | |
|---|---|---|
| No. | R′ | R″ |
| 39 | 3′-OCH₃ | 6-F |
| 40 | | 5-Cl |
| 41 | | 6-Cl |
| 42 | | 6-OCH₃ |
| 43 | 3′-OH, | 6-F |
| | 4′-OCH₃ | |
| 44 | 3′-OH, | 6-Cl |
| 45 | 4′-OCH₃ | 6-OCH₃ |
| 46 | 3′-CF₃ | 6-F |
| 47 | | 6-Cl |
| 48 | | 6-OCH₃ |

### General Procedure for Synthesis of Compounds 39-48

Equimolar amounts (0.5 mmol) of the oxindole and aldehyde were dissolved in ethanol (10 ml), a drop of piperidine (2 μl) was added and the mixture heated in a sealed vessel (10 ml) which was flushed with argon. The vessel was heated to 140° C. for 15 min in a microwave synthesizer (Biotage Initiator®). The reaction mixture was then cooled to room temperature and the resulting precipitate removed by filtration, carefully washed with cold ethanol and recrystallized at least one from ethanol to give the desired product.

### Assignment of Configuration of Synthesized Compounds

The synthesized compounds could exist as either E or Z isomers due to the presence of the exocyclic double bond. Thus, it was necessary to determine if synthesis had given rise to a single (predominant) isomer or to a mixture of isomers. Analyses of the ¹H and ¹³C NMR spectra of the compounds showed that they were obtained as a single (or predominant) isomer.

To determine if the synthesized compounds described herein were E or Z isomers, the chemical shifts of the aromatic protons on the benzylidene ring B (H-2′ and H-6′) were analyzed. In the Z but not E isomer, the H-2′ or H-6′ protons would be shifted downfield due to deshielding by the carbonyl group. In the literature, chemical shifts of 7.85-8.53 ppm were assigned to the Z isomer, and 7.45-7.84 ppm to the E isomer (Sun, L. et al., J Med Chem 1998, 41, 2588; Andreani, A et al, J Med Chem 2006, 49, 6922). For compounds already reported to be E isomers and whose chemical shifts were available from the literature, comparisons of our experimental values with reported values provided the needed confirmation (Corsico Coda, A.; et al, J. Chem. Soc., Perkin Trans. 2 (1972-1999), 1984, 4, 615; Andreani, A. et al, Eur. J. Med. Chem. 1990, 25, 187; Andreani, A., et al, Eur. J. Med. Chem. 1992, 27, 167).

For compounds whose stereochemistries have not been previously assigned, the chemical shifts of the H-2′ and H-6′ protons at 7.45-7.84 ppm were used to conclude that the synthesized compounds were E isomers. Compounds according to the invention such as compounds 46 and 47 were obtained as a mixture of E and Z isomers but with one predominant isomer. When the ¹H spectra of the isomers (after separation by column chromatography) were examined, the chemical shifts of H-2′ and H-6′ were found at 7.90-8.01 ppm for the predominant isomer, and 8.40-8.50 ppm for the minor isomer. Hence, the predominant isomer was assigned the E configuration.

### Experimental Procedures and Analytic Data for Compounds 39-48

(E)-6-Fluoro-3-(3-methoxybenzylidene)indolin-2-one (39), Yield 29%, mp 169-170° C.; MS-APCI: [M+H]⁺270.3 (269.1); ¹H NMR (300 MHz DMSO-d₆) δ ppm 3.79 (s, 3H), 6.68 (m, 2H), 7.03 (dd, J=8.1, 2.1 Hz, 1H), 7.24 (m, 2H), 7.43 (t, J=7.9 Hz, 1H), 7.54 (m, 2H), 10.77 (s, 1H); ¹³C NMR (75 MHz, DMSO-d6) δ ppm 168.92, 164.80, 161.55, 159.36, 144.94, 144.78, 135.60, 135.35, 135.31, 130.01, 126.75, 124.24, 124.10, 121.40, 117.35, 117.32, 115.69, 114.25, 107.74, 107.44, 98.39, 98.02, 55.24.

(E)-5-Chloro-3-(3-methoxybenzylidene)indolin-2-one (40), Yield 70%, mp 212-214° C.; MS-APCI: M+H]⁺286.2 (285.1); ¹H NMR (300 MHz DMSO-d₆) δ ppm 3.80 (s, 3H), 6.88 (d, J=8.7 Hz, 1H), 7.07 (m, 1H), 7.27 (m, 3H), 7.46 (m, 2H), 7.68 (s, 1H), 10.75 (s, 1H); ¹³C NMR (75 MHz, DMSO-d6) 5 ppm 168.31, 159.39, 141.77, 137.55, 135.37, 130.05, 129.64, 127.02, 124.94, 122.51, 122.02, 121.50, 116.15, 114.18, 111.54, 55.26.

(E)-6-Chloro-3-(3-methoxybenzylidene)indolin-2-one (41), Yield 71%, mp 198-200° C.; MS-APCI: M+H]⁺286.2 (285.1); ¹H NMR (300 MHz DMSO-d₆) δ ppm 3.79 (s, 3H), 6.91 (in, 2H), 7.04 (dd, J=8.3, 2.3 Hz, 1H), 7.24 (m, 2H), 7.43 (t, J=7.9 Hz, 1H), 7.52 (d, J=8.3 Hz, 1H), 7.63 (s, 1H), 10.76 (s, 1H); ¹³C NMR (75 MHz, DMSO-d6) δ ppm 168.60, 159.36, 144.33, 136.55, 135.50, 134.20, 130.02, 126.71, 123.77, 121.52, 120.95, 119.80, 115.91, 114.28, 110.14, 55.24.

(E)-6-Methoxy-3-(3-methoxybenzylidene)indolin-2-one (42)⁴⁷: Yield 31%, mp 161-163° C., lit. 162-163° C.; MS-APCI: M+H]⁺282.2 (281.1); ¹H NMR (300 MHz DMSO-d₆) δ ppm 3.77 (m, 6H), 6.44 (m, 2H), 7.01 (dd, J=8.1, 2.1 Hz, 1H), 7.22 (m, 2H), 7.41 (m, 2H), 7.49 (d, J=8.3 Hz, 1H), 10.54 (s, 1H); ¹³C NMR (75 MHz, DMSO-d6) δ ppm 169.29, 161.15, 159.29, 144.77, 136.06, 132.32, 129.83, 127.36, 123.80, 121.37, 115.20, 114.18, 113.63, 106.52, 96.50, 55.30, 55.17.

(E)-6-Fluoro-3-(3-hydroxy-4-methoxybenzylidene)indolin-2-one (43), Yield 33%, mp 225-227° C.; MS-APCI: M+H]⁺286.2 (285.1); ¹H NMR (300 MHz DMSO-d₆) δ ppm 3.83 (s, 3H), 6.69 (t, J=10.2 Hz, 2H), 7.05 (m, 1H), 7.16 (s, 2H), 7.47 (s, 1H), 7.71 (dd, J=8.1, 5.8 Hz, 1H), 9.41 (s, 1H), 10.70 (s, 1H); ¹³C NMR (75 MHz, DMSO-d6) δ ppm 169.32, 164.48, 161.22, 149.50, 146.51, 144.52, 144.35, 136.20, 136.16, 126.69, 124.27, 123.93, 123.81, 122.16, 117.69, 117.65, 116.14, 112.13, 107.50, 107.20, 98.18, 97.82, 55.66.

(E)-6-Chloro-3-(3-hydroxy-4-methoxybenzylidene)indolin-2-one (44), Yield 53%, mp 220-223° C.; MS-APCI: M+H]⁺302.2 (301.1); ¹H NMR (300 MHz DMSO-d₆) δ ppm 3.84 (s, 3H), 6.87 (d, J=1.9 Hz, 1H), 6.94 (dd, J=8.3, 1.9 Hz, 1H), 7.06 (d, J=9.0 Hz, 1H), 7.18 (m, 2H), 7.52 (s, 1H), 7.70 (d, J=8.3 Hz, 1H), 9.41 (s, 1H), 10.69 (s, 1H); ¹³C NMR (75 MHz, DMSO-d6) δ ppm 169.02, 149.72, 146.55, 143.94, 137.45, 133.55, 126.64, 124.14, 123.53, 122.43, 120.77, 120.17, 116.30, 112.12, 109.96, 55.68.

(E)-3-(3-Hydroxy-4-methoxybenzylidene)-6-methoxyindolin-2-one (45), Yield 38%, mp 184-186° C.; MS-APCI: M+H]⁺298.2 (297.1); ¹H NMR (300 MH DMSO-d₆) δ ppm 3.75 (s, 3H), 3.83 (s, 3H), 6.44 (m, 2H), 7.03 (d, J=8.3 Hz, 1H), 7.13 (d, J=10.6 Hz, 2H), 7.31 (s, 1H), 7.64 (d, J=8.3 Hz, 1H), 9.35 (s, 1H), 10.49 (s, 1H); ¹³C NMR (75 MHz, DMSO-d6) δ ppm 169.72, 160.78, 149.11, 146.45, 144.38, 133.30, 127.23, 125.04, 123.63, 121.90, 116.09, 114.07, 112.14, 106.42, 96.43, 55.67, 55.34.

(E)-6-Fluoro-3-(3-(trifluoromethyl)benzylidene)indolin-2-one (46), Yield 50%, mp 132-135° C.; MS-APCI: M+H]⁺308.1 (307.1); ¹H NMR (300 MHz DMSO-d₆) δ ppm 6.66 (m, 2H), 7.32 (dd, J=8.3, 5.7 Hz, 1H), 7.64 (s, 1H), 7.77 (m, 2H), 7.97 (d, J=6.4 Hz, 2H), 10.82 (s, 1H); ¹³C NMR (75 MHz, DMSO-d6) δ ppm 168.62, 165.06, 161.79, 145.30, 145.13, 135.49, 133.38, 133.34, 132.87, 130.27, 129.96, 129.84, 129.42, 129.00, 128.10, 128.08, 125.99, 125.95, 125.80, 125.76, 123.91, 123.78, 122.16, 118.55, 117.00, 116.96, 107.77, 107.48, 98.57, 98.21. 46(Z), ¹H NMR (300 MHz DMSO-d₆) δ ppm 6.66 (dd, J=9.2, 2.1 Hz, 1H), 6.83 (m, 1H), 7.73 (m, 3H), 7.89 (s, 1H), 8.46 (d, J=7.5 Hz, 1H), 8.83 (s, 1H), 10.82 (s, 1H).

(E)-6-Chloro-3-(3-(trifluoromethyl)benzylidene)indolin-2-one (47)⁴⁸: Yield 51%, mp 204-207° C.; ¹H NMR (300 MHz DMSO-d₆) δ ppm 6.87 (d, J=7.9 Hz, 2H), 7.29 (d, J=7.9 Hz, 1H), 7.76 (m, 3H), 7.97 (d, J=7.2 Hz, 2H), 10.81 (s, 1H); ¹³C NMR (75 MHz, DMSO-d6) δ ppm 168.26, 144.60, 135.39, 134.66, 132.94, 130.02, 129.84, 129.42, 128.06, 126.18, 126.15, 125.94, 125.90, 125.75, 123.46, 122.14, 121.01, 119.44, 110.33. 47(Z), ¹H NMR (300 MHz DMSO-d₆) δ ppm 6.88 (s, 1H), 7.05 (dd, J=8.1, 1.7 Hz, 1H), 7.72 (m, 3H), 7.91 (s, 1H), 8.43 (d, J=7.9 Hz, 1H), 8.84 (s, 1H), 10.81 (s, 1H).

(E)-6-Methoxy-3-(3-(trifluoromethyl)benzylidene)indolin-2-one (48), Yield 43%, mp 157-160° C.; MS-APCI: M+H]⁺320.1 (319.1); ¹H-NMR (300 MHz DMSO-d₆) δ ppm 3.74 (s, 3H), 6.40 (dd, J=10.9, 2.6 Hz, 2H), 7.28 (d, J=8.3 Hz, 1H), 7.47 (s, 1H), 7.74 (ddd, J=15.0, 7.9, 7.6 Hz, 2H), 7.96 (d, J=7.2 Hz, 2H), 10.61 (s, 1H); ¹³C NMR (75 MHz, DMSO-d6) δ ppm 169.04, 161.54, 145.14, 135.99, 132.91, 130.40, 130.16, 129.90:129.74, 129.44, 129.31, 128.89, 128.72, 125.83, 125.69, 125.65, 125.59, 123.50, 122.22, 118.61, 113.28, 106.61, 96.75, 55.39.

### Example 2

### Determination of Compound Purity by HPLC for Compounds 39-48

The purity of compounds was assessed by reverse phase high pressure column chromatography. Determinations were carried out on a Waters Delta 600 liquid chromatography system, using Agilent Zorbax Eclipse XDB-C₁₈ column (4.6 mm×150 mm, 5 μm). The isocratic mode was employed using two solvent systems (A: methanol-water and B: acetonitrile-water in the ratio of 4:1). Flow rate was fixed at 1 ml/min and UV detection at dual wavelengths (278 nm and 305 nm) was employed. The area under the main peak was determined and expressed as a % of total peak area during a 20 min run. All compounds were purified until their chromatograms showed a main peak (P_{HPLC}) with areas >96% on both solvent systems. The retention time (t_{R}) of the principal peak in two solvent system was determined in minutes (min).

TABLE 2
Determination of compound purity by HPLC

| | |
|---|---|
| Solvent system A | Solvent system B |

| Compound | t_{R}(min) | P_{HPLC} | t_{R}(min) | P_{HPLC} |
|---|---|---|---|---|
| 39 | 3.9 | 97.4% | 2.4 | 98.0% |
| 40 | 5.4 | 98.3% | 2.9 | 97.9% |
| 41 | 5.9 | 97.5% | 2.9 | 98.0% |
| 42 | 3.7 | 98.5% | 2.2 | 98.3% |
| 43 | 2.2 | 99.6% | 1.8 | 98.7% |
| 44 | 2.8 | 99.1% | 1.9 | 97.5% |
| 45 | 2.1 | 98.0% | 1.7 | 96.6% |
| 46 | 5.0 | 96.5% | 2.8 | 99.1% |
| 47 | 7.1 | 98.2% | 3.4 | 97.4% |
| 48 | 4.8 | 98.4% | 2.7 | 96.8% |

### Materials for Biological Assays

Menadione, digitonin, 3-(4,5-dimethylthiazol-2-yl)-2,5-diphenyltetrazolium bromide (MTT), β-naphthoflavone (BNF), dicuomarol, salicylamide, propidium iodide, RNase A, 7-ethoxyresorufin and sulforaphane were purchased from Sigma-Aldrich (St Louis, Mo.). 2,3,7,8-Tetrachlorodibenzo-p-dioxin (TCDD) was purchased from AccuStandard (New Haven, USA). Other reagents were of analytical grade.

### Example 3

### Determination of NQO1 Activity

Hepa1c1c7 were purchased from American Type Culture Collection (Rockville, Md.). and cultured in alpha-minimum essential medium (α-MEM) without nucleosides, and containing 10%(v/v) heat- and charcoal-treated fetal calf serum (1 g of charcoal per 100 ml of serum; 90 mM at 55° C.), 0.15% sodium bicarbonate, 0.01% penicillin G, 0.01% streptomycin sulfate in an humidified atmosphere of 5% CO₂ at 37° C. Cells were sub cultured when they reached 80-90% confluency and used within 6-17 passages for determinations. For the assay, about 10000 cells were grown in each well of a 96-well plate for 24 h in α-MEM. Total protein content per was found to be 0.036 mg per well based on the Bradford assay. Stock solutions of test compounds were prepared in DMSO and aliquots were added to each well to give the desired concentration. The final concentration of DMSO in each well was kept at 0.5% v/v or lower. After incubation for 48 h, the media was decanted and the cells lyzed by a solution containing 0.8% w/v digitonin and 2 mM EDTA with incubation at 37° C. (10 mM). The plates were gently agitated on an orbital shaker for 10 mM at 23° C. An aliquot (2000) of a solution (“complete reaction mixture”) was then added to each well. This solution was freshly prepared just prior to use and consisted of 7.5 ml 0.5M Tris-Cl (pH=7.4),100mg bovine serum, 1 ml 1.5% Tween-20, 0.1 ml 7.5 mM FAD, 1 ml 150 mM glucose 6-phosphate, 90 μl mM NADP, 300 units yeast glucose-6-phosphate dehydrogenase, 45 mg MTT and deionized water made up to a final volume of 150 ml. Menadione (1 μl of 50 mM menadione dissolved in acetonitrile for every milliliter of reaction mixture was added just before the mixture was dispensed into the wells. After addition, the plate was gently agitated for 5 min and the reaction was quenched by addition of a solution (50 μl) of 0.3 mM dicoumarol in 0.5% DMSO and 5 mM potassium phosphate, pH=7.4. A blue color due to the formation of formazan was observed in each well, the absorbance of which was measured at 590 nm on a plate reader. Blank wells contained no cells and control wells contained Hepa1c1c7 cells treated with medium containing 0.5% DMSO but without the test compound. NQO1 induction activity of a compound tested at a given concentration was determined from the equation:

Degree of induction=A_{test compound}−A_{blank}/A_{control}−A_{blank}

where A is absorbance of formazan measured at 590 nm.

Sulforaphane and BNF were determined under similar conditions as positive controls. The results were plotted (degree of induction versus concentration) with OriginPro 7.5 SR1 (Version V7.5776 B776), OriginLab Corporation, MA and the concentration of test compound required to double the basal NQO1 activity (CD) was determined. Three separate determinations were made and CD was reported as mean±SD for n=3.

### Induction of NQO1 in Hepa1c1c7 Cells

The compounds were screened for induction of NQO1 activity in the murine hepatoma (Hepa1c1c7) cells (Prochaska, H. J.; Santamaria, A. B. Anal. Biochem. 1988, 169, 328). Briefly, the assay is based on the generation of NADPH when glucose-6-phosphate (G6P) is reduced by G6P dehydrogenase in the presence of its cofactor NADP. NADPH serves as an electron donor for the NQO1 mediated reduction of menadione (a quinone) to menadiol (a diphenol). The latter reduces MTT to formazan (a colored product) and its formation is monitored in the assay. A compound that induces NQO1 increases the rate at which menadiol is formed, and hence the generation of formazan.

The compounds were first screened for their growth inhibitory IC₅₀ values on the Hepa1c1c7 cells to ensure that concentrations used in the induction assay were not cytotoxic to the cells. Compounds that did not affect cell viability at 25 μM (highest concentration tested) were listed as having IC₅₀ values exceeding 25 μM. The compounds were tested over a 10-100 fold concentration range for induction activity, which was expressed in terms of the CD, defined as the concentration required to bring about a 2-fold increase in NQO1 activity of treated cells compared to untreated Hepa1c1c7 cells. The results are given in Table 3. Positive controls for this assay were the known NQO1 inducers sulforaphane and β-napthoflavone (BNF). Their CD values were 0.26 (±0.04) μM and 0.028 (±0.003) μM, which agreed well with reported values (Dinkova-Kostova, A. T.; Liby, K. T.; Stephenson, K. K.; Holtzclaw, W. D.; Gao, X.; Suh, N.; Williams, C.; Risingsong, R.; Honda, T.; Gribble, G. W.; Sporn, M. B.; Talalay).

TABLE 3
CD and IC50 values of compounds 39-48 on Hepa1c1c7 cells

| | | | QR Induction of | IC₅₀ (μM) on |
|---|---|---|---|---|
| | Ring A | Ring B | Hepa1c1c cells ^{a} | Hepa1c1c7 |
| NO. | (R¹) | (R²) | CD(μM) | cells |
| 39 | 6-F | 3′-OCH₃ | 0.17 ± 0.05 | 9.8 |
| 40 | 5-Cl | 3′-OCH₃ | 0.36 ± 0.06 | 17.5 |
| 41 | 6-Cl | 3′-OCH₃ | 0.039 ± 0.01 | >25 |
| 42 | 6-OCH₃ | 3′-OCH₃ | 0.13 ± 0.05 | 9.8 |
| 43 | 6-F | 3′-OH, 4′-OCH₃ | 0.11 ± 0.05 | 15.6 |
| 44 | 6-Cl | 3′-OH, 4′-OCH₃ | 0.076 ± 0.01 | >25 |
| 45 | 6-OCH₃ | 3′-OH, 4′-OCH₃ | 0.24 ± 0.08 | 9.8 |
| 46 | 6-F | 3′-CF₃ | 0.016 ± 0.007 | 2.2 |
| 47 | 6-Cl | 3′-CF₃ | 0.0041 ± 0.0009 | 1.1 |
| 48 | 6-OCH₃ | 3′-CF₃ | 0.085 ± 0.014 | 2.6 |

^{a} Concentration required to bring about a 2-fold increase in the NQO1 activity of the treated cells compared to the untreated Hepa1c1c7 cells. Mean and SD (n = 3).

As can be seen in Table 3, the compounds of the invention were generally potent NQO1 inducers, with CD values ranging from 0.36 μM to 4 nM. To determine if substitution on ring A was instrumental in the improved induction profiles, the following comparisons (analyzed by one-way ANOVA) were made. In the case of compounds 39-42 (3′-OCH₃ on ring B, various substituents on ring A), greater induction was observed for 41 (6-Cl) and 42 (6-OCH₃). When comparing a compound in which 3′-CF₃ on ring B, no ring A substituent) with compounds 46-47 (3′-CF₃ on ring B, various ring A substituents), only 47 (6-C1) improved activity. Thus, compounds with 6-Cl on ring A fared better than their unsubstituted ring A analogues only when coupled with certain ring B groups, namely 3′-OCH₃ and 3′-CF₃. This may be true for other substituents as well but it has presently not been determined whether ring A or B plays a dominant role.

### Example 4

### Determination of 7-ethoxyresorufin O-deethylase (EROD) activity in Hepa1c1c7 cells

Hepa1c1c7 cells were plated at a density of 10000 cells per well in a 96-well plate and cultured for 24 h. A stock solution of test compound was prepared in DMSO and serially diluted with medium to give the desired concentration in the well. The final concentration of DMSO was 0.5% v/v. The cells were incubated with test compound for 48 h, after which the medium was removed, the well washed with 200 μl of 1×phosphate-buffered saline solution (PBS) and then incubated with 5 μM 7-ethoxyresorufin and 2 mM salicylamide in 200 μl of medium at 37° C., 40 min. Readings were taken at λ_{excitation} of 530 nm and λₑₘᵢₛₛᵢₒₙ of 590 nm on a fluorometer. Fluorescence (if any) of the test compound was determined at the same wavelengths to take into account its contribution to the observed readings. Readings of empty wells (no cells, “blank”) and wells with Hepa1c1c7 cells in medium containing 0.5% DMSO but without test compound (“control”) were also determined. TCDD, a strong inducer of CYP1A1 activity, as well as BNF and sulforaphane were used as controls.

CYP1A1 induction activity was given by the expression:

Degree of induction=F_{Cells+test compound}−F_{Blank}/F_{Control}−F_{Blank}

### Effect of compounds on 7-ethoxyresorufin O-deethylase (EROD) activity in Hepa1c1c7 Cells

Since induction of NQO1 activity was observed for the compounds mentioned above (see Example 3), it was important to determine if phase 1 enzyme activity was induced as well. The EROD assay was used for this purpose (Burke, M. D.; Mayer, R. T., Drug Metab Dispos 1974, 2, 583). EROD activity describes the rate at which CYP1A1 caused the O-deethylation of 7-ethoxyresorufin to resorufm. If a compound induced CYP1A1 activity, more resorufm would be formed compared to control untreated Hepa1c1c7 cells and the ratio of resorufin fluorescence in treated versus control Hepa1c1c7 cells would increase. This ratio was determined for compounds at concentrations that were approximately 4 times their CD values. A fixed concentration was not used here because it would be difficult to select this concentration in view of the large variation in NQO1 induction activities. The quotient of NQO1 and CYP1A1 induction ratios (ratio determined at the same concentration) was also obtained for each compound. Values >1 would imply greater induction of NQO1 relative to CYP1A1. The results for compounds 39-48 are tabulated in Table 4.

TCDD, BNF and sulforaphane were used as controls in this experiment. TCDD is a known inducer of CYP1A1 (Nishiumi, S.; Yamamoto, N.; Kodoi, R.; Fukuda, I.; Yoshida, K.; Ashida, H., Arch Biochem Biophys 2008, 470, 187) and when used in this assay, gave a ratio of 2.99 in the EROD assay at 1 nM. BNF and sulforaphane are bifunctional and monofunctional NQO1 inducers respectively (Dinkova-Kostova, A. T.; Liby, K. T.; Stephenson, K. K.; Holtzclaw, W. D.; Gao, X.; Suh, N.; Williams, C.; Risingsong, R.; Honda, T.; Gribble, G. W.; Sporn, M. B.; Talalay). Hence, BNF should induce both CYP1A1 and NQO1 activities in Hepa1c1c7 cells, whereas sulforaphane should induce NQO1 activity to a greater degree than CYP1A1 activity. It was found that BNF at 0.01 μM induced NQO1 activity by 1.83 times (NQO1 induction ratio) and CYP 1A1 activity by 1.13 times (CYP1A1 induction ratio), thus giving an NQO1/CYP1A1 quotient of 1.62. The results obtained with sulforaphane were in keeping with its monofunctional character. At 1 μM, it barely induced CYP1A1 activity (ratio=0.924) but increased NQO1 activity by nearly four fold (ratio=3.89).

In general, the compounds of the invention were weak inducers of CYP1A1. As an illustrative example, compound 47 shows increasing CYP1A1 activity by more than 2 fold. More important than the CYP1A1 induction ratio was the quotient of NQO1 versus CYP1A1 induction ratios as this value gave an indication of whether the compound preferentially induced NQO1 to CYP1A1. Strong induction of CYP1A1 may not be unfavourable if it is counter-balanced by even stronger induction of NQO1. The most potent NQO1 inducer was 47 (NQO1 induction ratio 5.20, Table 4) and it had a modest NQO1/CYP1A1 quotient of 2.17. Similar correlations were noted for compound 45.

TABLE 4
CYP1A1 and NQO1 induction ratios of compounds 39-48

| | | | CYP1A1 | NQO1 | |
|---|---|---|---|---|---|
| | Ring A | Ring B | induction | induction | NQO1/CYP1A1 |
| Number ^{a} | (R¹) | (R²) | ratio ^{b} | ratio ^{c} | Quotient ^{d} |
| 39 (1 μM) | 6-F | 3′-OCH₃ | 1.25 ± 0.06 | 3.21 | 2.57 |
| 40 (2.5 μM) | 5-Cl | 3′-OCH₃ | 1.32 ± 0.08 | 4.34 | 3.29 |
| 41 (0.1 μM) | 6-Cl | 3′-OCH₃ | 1.51 ± 0.13 | 2.56 | 1.70 |
| 42 (0.5 μM) | 6-OCH₃ | 3′-OCH₃ | 1.13 ± 0.01 | 3.08 | 2.72 |
| 43 (0.5 μM) | 6-F | 3′-OH, 4′-OCH₃ | 2.05 ± 0.07 | 4.10 | 2.00 |
| 44 (1 μM) | 6-Cl | 3′-OH, 4′-OCH₃ | 2.33 ± 0.13 | 5.09 | 2.18 |
| 45 (1 μM) | 6-OCH₃ | 3′-OH, 4′-OCH₃ | 1.05 ± 0.01 | 4.34 | 4.13 |
| 46 (0.05 μM) | 6-F | 3′-CF₃ | 1.23 ± 0.08 | 3.31 | 2.69 |
| 47 (0.05 μM) | 6-Cl | 3′-CF₃ | 2.40 ± 0.27 | 5.20 | 2.17 |
| 48 (0.5 μM) | 6-OCH₃ | 3′-CF₃ | 1.36 ± 0.07 | 4.22 | 3.10 |

^{a} Compound number and concentration used in CYP1A1 induction assay (≈4 × CD for NQO1 activity) is given in parentheses.
^{b} Determined on Hepa1c1c7 cells at concentrations given in column 1. Mean ± SD, n = 3. CYP1A1 induction ratio = (Fluorescence_{Cells + Test Compound} − F_{Blank})/(Fluorescence_{Control Cells} − F_{Blank})
^{c} Determined on Hepa1c1c7 cells at the same concentration for CYP1A1 induction ratio Values were read off from plot of degree of induction versus concentration for each compound.
^{d} Quotient = NQO1 induction activity/CYP1A1 induction activity
^{j} This value was read off from a curve constructed with concentrations 0.005, 0.05, 0.5 and 5 μM BNF. Therefore no SD is assigned to this value. At 0.05 μM BNF, CYP1A1 ratio was 1.27 ± 0.12.

### Example 5

### Determination of Antiproliferative Activity by the Microculture Tetrazolium (MTT) Assay

The antiproliferative activity of the compounds according to the invention was determined by the MTT assay on the following cell lines: MCF7 (human breast cancer cell line), HCT116 (human colon cancer cell line), and CCL186 (normal human diploid embryonic lung fibroblast). The cell lines were purchased from American Type Culture Collection (Rockville, Md.). MCF7 and CCL186 cells were cultured in Eagle's Minimum Essential Medium (EMEM) with 1 mM sodium pyruvate, 1.5 g/L sodium bicarbonate, 2 mM L-glutamine supplemented with 10% FBS and 0.01% antibiotics. HCT116 cells were cultured in McCoy's 5A Medium with 2.2 g/L sodium bicarbonate supplemented with 0% FBS and 0.01% antibiotics. Cells were plated at the following densities: 10000 cells/well (MCF7), 4000 cells/well (HCT116), 5000 cells/well (CCL186). They were grown for 24 h in a 96-well plate, with 100 μl of corresponding medium in each well. The test compounds were prepared in DMSO and diluted to a series of concentrations with medium. Not more than 1% DMSO (final concentration) was present in each well. The test compounds were incubated with the cells for 72 h, after which 100 μl MTT solution (0.5 mg/ml in 1× PBS) was added for 3 h and the cells lysed to release the formazan product. The latter was dissolved in DMSO (150 μl) and absorbance determined within 30 min at 590 nm on a microtitre plate reader. Cell survival was given by the expression:

Cell survival (%)=[(A_{cells+test compound}−A_{blank})/(A_{untreated cells}−A_{blank})]×100

where A is the absorbance of formazan measured at 590 nm in the test (A_{cells+test compound}), control (A_{untreated cells}) or blank (A_{blank}) wells. Each concentration of test compound was evaluated on 3 separate occasions. The concentration (IC₅₀) that inhibited 50% of cell growth was determined from the sigmoidal curve obtained by plotting % surviving cells versus concentration using OriginPro 7.5 SR1 (Version V7.5776 B776), OriginLab Corporation, MA. The MTT assay was also carried out on Hepa1c1c7 cells for the purpose of determining cytotoxicity of test compounds. The same procedure was adopted except that the incubation period of compound with cells was 48 h, instead of 72 h.

### Antiproliferative Activity of Compounds on HCT116, MCF7 and CCL186 Cells

The compounds were initially screened at a fixed concentration of 10 μM on the human cancer cell lines HCT116 and MCF7 cells. Compounds such as 42, 45-48 caused cell death by more than 50% at this concentration and these compounds were further evaluated for their IC₅₀ on the two cancer cell lines (Table 5). The small number (11, 18%) of compounds found to affect cell viability may imply that more stringent requirements exist for antiproliferative activity, as compared to chemoprevention where most compounds induced NQO1 activity with CD values <10 μM (Table 3).

TABLE 5
IC₅₀ and Selectivity Ratios of the following compounds on human
breast cancer (MCF-7), human colon cancer (HCT116) and normal
human diploid embryonic lung fibroblast (CCL-186) cell lines

| | Ring A | Ring B | | IC₅₀ (μM)^{a} | |
|---|---|---|---|---|---|
| Compound | (R) | (R′) | MCF-7 | HCT-116 | CCL-186 |
| 42 | 6-OCH₃ | 3′OCH₃ | 1.2 ± 0.4 | 1.3 ± 0.4 | 1.8 ± 0.2 |
| | | | [symbol] | [symbol] | |
| 45 | 6-OCH₃ | 3′OH-4′-OCH₃ | 3.7 ± 0.3 | 7.8 ± 0.9 | 11.3 ± 0.4 |
| | | | [symbol] | [symbol] | |
| 46 | 6-F | 3′-CF₃ | 8.4 ± 0.3 | 8.9 ± 0.3 | 7.5 ± 0.7 |
| | | | [symbol] | [symbol] | |
| 47 | 6-Cl | 3′-CF₃ | 3.8 ± 0.3 | 7.5 ± 0.4 | 6.6 ± 0.5 |
| | | | [symbol] | [symbol] | |
| 48 | 6-OCH₃ | 3′-CF₃ | 1.9 ± 0.5 | 3.8 ± 0.6 | 5.4 ± 0.3 |
| | | | [symbol] | [symbol] | |

^{a}Concentration required to reduce cell survival by 50% after 72 h incubation with test compound. Mean ± SD, n = 3. Values in bold and italics represent the selectivity ratio: IC_{50 CCL186}/IC_{50 MCF7} or IC_{50 CCL186}/IC_{50 HCT116}

The compounds in Table 5 had comparable antiproliferative activities on both cancer cell lines, with IC₅₀ values ranging from 1.2 to 19.6 μM. When these activities were compared to those obtained on a normal cell line (human lung fibroblasts), modest selectivities (approximately 2-fold on average) were observed. Compounds such as 46 and 47 did not discriminate much between cancerous and normal cells and had selectivity ratios that were close to 1.

The compounds according to the invention are strongly represented for antiproliferative activity. As an illustrative example, compounds with 3′-CF3 in ring B (46, 47, 48) were listed in Table 5, indicating the important role played by this substituent, quite independently of the ring A substituent. Similarly, ring A of Class 2 had only 3 different substituents at position 6, namely 6-OCH₃, 6-Cl and 6-F. Of these, 6-OCH₃ appears to be preferred because all three compounds (42, 45, 48) with 6-OCH₃ are listed in Table 5, irrespective of the group on ring B. Hence, it may be concluded that 6-OCH₃ (ring A) and 3′-CF₃ (ring B) are preferred groups for antiproliferative activity, notwithstanding that the most active compound 42 (6-OCH₃, 3′-OCH₃) did not have both features in the same molecule. The substituent preferences for antiproliferative activity are comparatively well defined, unlike NQO1 induction activity as described in Example 3.

### Example 6

### Determination of the Effects of Test Compounds on the Cell Cycle of HCT116 Cells by Cell Cytometry

HCT116 cells were grown to confluence and maintained in this state for at least 5 days without change of media. The serum starved cells were then trypsinized and subcultured at densities of 5×10⁵ cells/well in six-well plates. Growth media contained 10% fetal calf serum. The test compound was added either immediately to cells synchronized at G1 phase, or 24 h later when cells were synchronized at G2 phase. The cells were incubated with test compound for 24 h from the time of addition. After this time, the cells were harvested, trypsinized and fixed in 70% ice-cold ethanol for a minimum of 24 h. After centrifugation, the supernatant was discarded and the pellet was treated with RNase A (200 μg/ml) for 30 min at room temperature, followed by cell staining using propidium iodide at a final concentration of 20 μg/ml. The stained cells were then analyzed for cell cycle distribution in the sub-G1, G1, S and G2/M phases on a Dako Cytomation Cyan LX (Dako Colorado, Fort Collins, Colo., USA) equipped with an argon solid state laser (488 nm) using the Summit (version 4.3) software. Compounds were tested at a concentration of ≈1.5×IC₅₀, with IC₅₀ determined by the MTT assay on HCT116 cells.

### Statistical Analysis

Data was analyzed for statistical significance by one-way ANOVA followed by Tukey HSD as post-hoc test on SPSS 15.0 for Windows, Chicago, Ill. Spearman's correlation analysis was carried out on the same software. A level of probability of 0.05 was used as the criterion for significance.

### Effect of Selected Compounds on Cell Cycle of HCT116 Cells

To have a better understanding of the mechanisms underlying the antiproliferative activities of the compounds listed in Table 5, their effects on the cell cycle was investigated by fluorescence-activated cell sorter (FACS) analysis using flow cytometry. The experiments were designed to examine the effects of the test compounds on synchronized cell populations at the GI or G2/M phase. G1 synchronized cells were obtained by growing cells to confluence for up to 5 days and then stimulating their re-entry into the cell cycle by sub-culturing at lower densities. After 24 hours of growth, these cells would be aligned at the G2/M phase.

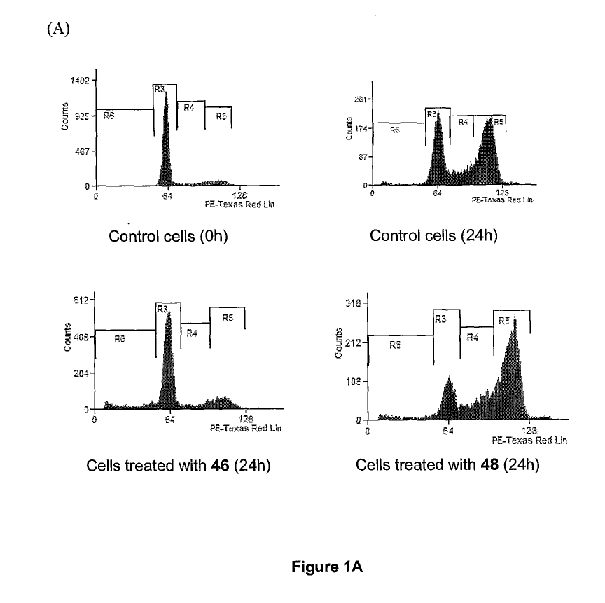

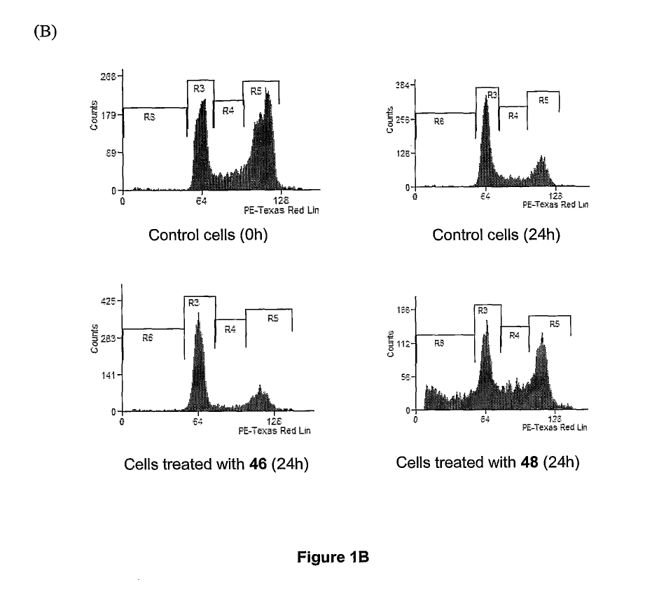

Cells released from the G1-block were exposed to a fixed concentration (≈1.5×IC₅₀ on HCT116 cells) of test compound for 24 h, after which the distribution of cells at the different phases were determined, and compared to control untreated cells. Cells aligned at the G2/M phase were similarly treated. Representative FACS diagrams of control cells released from G1 block and G2 block are given in FIG. 1. The proportion of cells in each phase after release from G1- or G2-arrest is given in Table 6.

TABLE 6
Effect of Test compounds on various phases of cell cycle

| | | | | |
|---|---|---|---|---|
| Compound ^{b} | Sub-G1 | G1 | S | G2/M |

HCT 116 cells released from G1 Block ^{a}

| | | | | |
|---|---|---|---|---|
| Control (0 h) | 0.46 | 80.19 | 6.8 | 11.57 |
| Control (24 h) | 1.94 | 34.03 | 14.90 | 49.47 |
| 45 (15 μM) | 5.26 | 17.49 | 21.13 | 56.49 |
| 46 (15 μM) | 12.68 | 64.93 | 8.04 | 14.58 |
| 47 (15 μM) | 12.39 | 65.55 | 7.10 | 15.13 |
| 48 (5 μM) | 4.39 | 17.50 | 19.12 | 59.16 |

HCT 116 cells released from G2 Block ^{a}

| | | | | |
|---|---|---|---|---|
| Control (0 h) | 1.17 | 31.12 | 12.11 | 55.97 |
| Control (24 h) | 1.24 | 55.88 | 14.93 | 27.90 |
| 42 (2.5 μM) | 30.54 | 26.01 | 15.36 | 43.57 |
| 45 (15 μM) | 15.54 | 26.01 | 15.36 | 43.57 |
| 46 (15 μM) | 2.49 | 64.15 | 8.82 | 25.00 |
| 47 (15 μM) | 10.62 | 53.47 | 8.05 | 28.39 |
| 48 (5 μM) | 20.69 | 30.55 | 17.96 | 31.58 |

^{a} Concentration used is indicated in brackets. ≈1.5 × IC_{50 HCT116}
^{b} Proportion of cells in each phase was the mean of 3 separate determinations.

Analysis of the results revealed that the compounds caused either G1 arrest (46, 47) or G2 arrest (42, 45, 48). Its antiproliferative activity may arise from other mechanisms like autophagy or necrosis. Taking 46 as an example of a compound associated with G1 arrest, the FACS diagram showed that when cells released from G1 block were treated with 46, the progression from G1 to G2/M was interrupted (FIG. 1A). Notable increases in the sub-G1 phase were observed (Table 6). As anticipated, 46 had no effect on the transition of cells from G2/M to GI (FIG. 1B). In the case of 48 which caused G2-arrest, it did not interfere with the progression of cells released from G1 (FIG. 1A) but prevented cell transit from G2/M to G1 (FIG. 1B). Thus, the proportion of cells in G1 did not increase after exposure (24 h) to 48 (30.55% in G1, compared to 55.88% in untreated cells) (Table 6). There were also significant increases in apoptotic cells in the sub-G1 phase (FIG. 1B, Table 6).

A structural trend was observed among the compounds that caused G1 or G2 arrest. Compounds (42, 45, 48) that caused G2 arrest had in common the presence of one or two methoxy groups on ring A or B. Compounds 42, 45 and 48 had 6-OCH₃ on ring A. Rings B of 42 and 45 were also methoxylated. By contrast, compounds (46, 47) that caused G1 arrest were halogentated (46, 47).

### Example 7

### Anti-Proliferative Potential of Compounds 39-48 in HCC Cell Lines

The anti-cancer potential of these compounds against HCC were tested using a phenotypic assay where compounds were screened over 100-fold concentration range (0.1 to 10 μM) using two representative liver cancer cell lines, HepG2 and HuH7. Additionally, THLE2 was employed as normal hepatocyte cell line to differentiate anti-cancer potential from non-specific cytotoxicities. IC₅₀ values based on 50% inhibition of cell viability at 72 h were determined by reduction in intracellular ATP content. From the results, Compound 47 exhibited submicromolar IC₅₀ against both HuH7 and HepG2 (0.5 μM and 0.6 μM respectively). Compound 46 and 48 displayed similar potency but more pronounced towards HepG2 than HuH7 (Table 2). These responses were either comparable or superior to the clinically relevant indolinone, sunitinib, with IC₅₀ of 4.7 μM and 4.5 μM in HuH7 and HepG2 respectively.

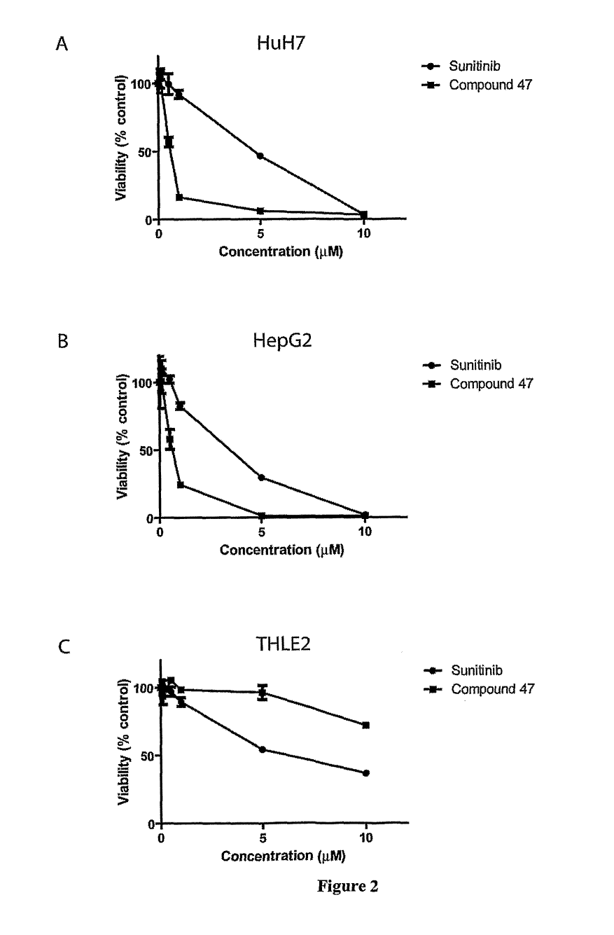

Cell viability assay shown in FIG. 2 was analyzed by Prism5 for the determination of respective IC₅₀ values.

TABLE 7
IC₅₀ of test compounds based on viability
assay in HuH7, HepG2 and THLE2

| | IC₅₀ HuH7 | IC₅₀ HepG2 | IC₅₀ THLE2 |
|---|---|---|---|
| Compound No. | (μM) | (μM) | (μM) |
| 39 | >10 | 7.6 | >10 |
| 40 | >10 | 9.2 | >10 |
| 41 | >10 | 8.5 | >10 |
| 42 | 4.0 | 2.2 | 1.2 |
| 43 | >10 | 1.2 | >10 |
| 44 | >10 | 1.0 | >10 |
| 45 | 8.0 | 1.4 | >10 |
| 46 | 1.1 | 0.4 | >10 |
| 47 | 0.5 | 0.6 | >10 |
| 48 | 2.3 | 0.4 | 3.6 |
| Sunitinib | 4.7 | 4.5 | 4.5 |

Subsequently, the safety index of tested compounds was quantified by normalizing the percentage viability at 10 μM with the normal liver control cell line (i.e. THLE2/(HuH7 or HepG2). Most compounds gave a value >1, indicating a selectivity of anti-proliferative effect for the compounds in tumor vs. normal cells (Table 8).

Cell viability was determined as described previous in Table 7. Percentage viability at 10 μM treatments was determined and safety ratios were calculated by normalizing survival in HCC cell lines to THLE2.

TABLE 8
Safety ratio of test compounds at 10 μM in HCC and normal liver cell lines

| Compound No. | HuH7 | SR_{(HuH7/THLE2)} | HepG2 | SR_{(HepG2/THLE2)} |
|---|---|---|---|---|
| 39 | 65.8 ± 1.1 | 1.19 | 40.0 ± 4.2 | 1.95 |
| 40 | 57.5 ± 6.6 | 1.55 | 46.1 ± 2.2 | 1.93 |
| 41 | 101.7 ± 6.9 | 0.87 | 46.4 ± 2.5 | 1.90 |
| 42 | 49.2 ± 4.0 | 0.79 | 20.7 ± 2.0 | 1.89 |
| 43 | 84.1 ± 7.5 | 1.22 | 28.3 ± 6.7 | 3.63 |
| 44 | 63.0 ± 4.0 | 1.36 | 15.0 ± 1.3 | 5.72 |
| 45 | 46.6 ± 1.8 | 1.54 | 19.9 ± 1.4 | 3.61 |
| 46 | 11.3 ± 1.0 | 6.39 | 4.2 ± 0.3 | 17.19 |
| 47 | 3.3 ± 0.2 | 21.76 | 1.1 ± 0.09 | 65.27 |
| 48 | 27.3 ± 0.5 | 1.27 | 14.5 ± 1.3 | 2.40 |
| Sunitinib | 3.4 ± 0.2 | 10.79 | 1.8 ± 0.3 | 20.39 |

Remarkably, Compound 47 demonstrated a pronounced safety as assessed by both HuH7 (SR=21.76) and HepG2 (SR=65.27). Comparison with sunitinib confirmed that Compound 47 exhibited better efficacy and reduced cytotoxicity (FIG. 2).

### Example 8

### Effects of Compound 47 on a Separate Panel of HCC Cell Lines

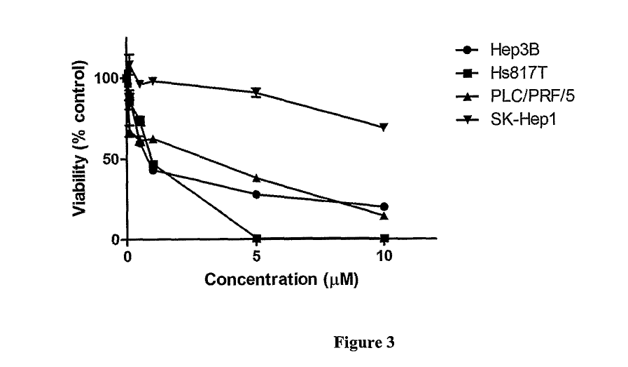

Accordingly, Compound 47 was screened against a larger panel of 4 additional HCC cell lines. These subsequent experiments corroborated its effectiveness with IC₅₀ in low micromolar or submicromolar concentrations in 3 of the 4 cell lines tested. SK-Hep1 was the only marginal responder where IC₅₀ was not attainable at 10 μM. Hs817T on the other hand was the most sensitive to Compound 47 with undetectable viability at 5 μM (FIG. 3).

### Example 9

### Effects of Compound 47 on Biochemical Markers of Cell Cycling and Apoptosis

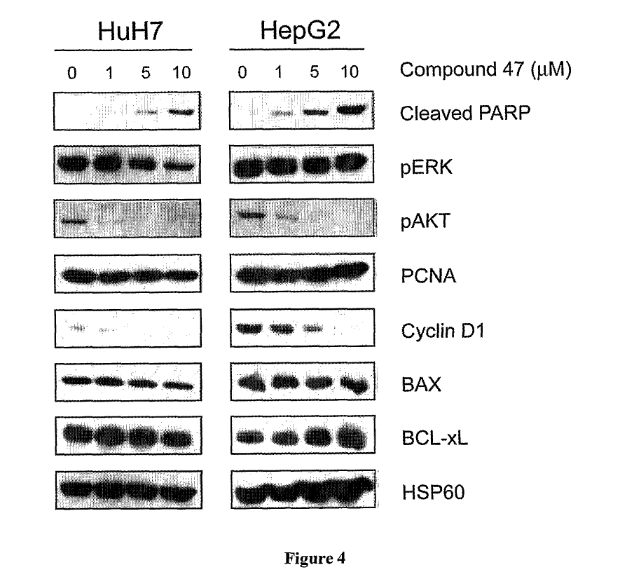

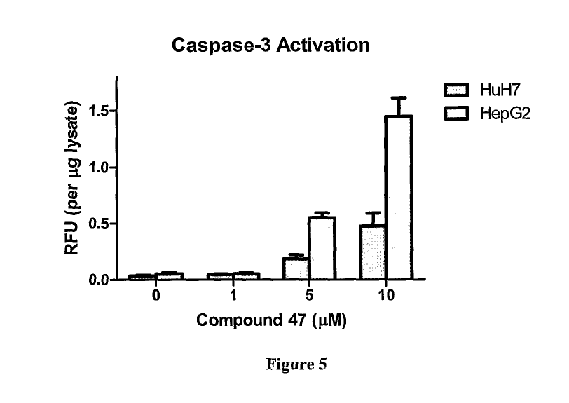

Next, the mechanism of Compound 47's anti-proliferative effects was investigated. Compound 47 invoked a dose-dependent inhibition of both Erk and Akt phosphorylation at 24 h (FIG. 3). There was also a corresponding reduction of cell cycling markers, cyclin-D1 and PCNA expression. Similarly, apoptotic markers like cleaved PARP (FIG. 4) and caspase-3 activation (FIG. 5) confirmed the participation of pro-apoptotic mechanism. However, there was little effect on the mitochondrial apoptotic players Bax and BCL-xL.

### Example 10

### Compound 47 Inhibits AFP Transcription in HuH7

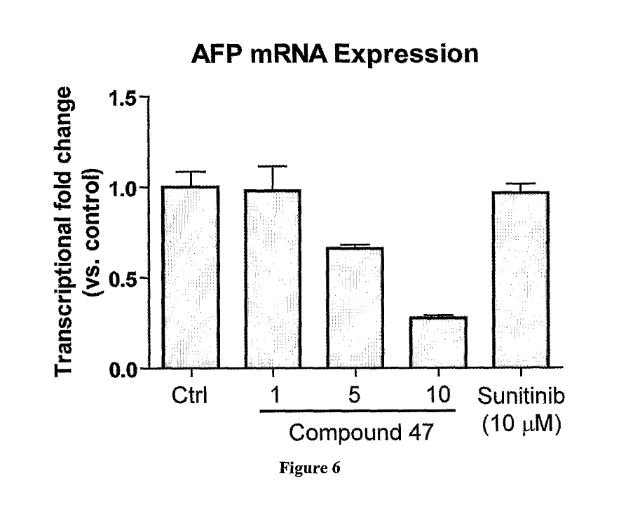

To evaluate the specificity of Compound 47 towards treatment of HCC, tumor marker AFP was measured transcriptionally. The advantage of AFP mRNA quantification over the more established ELISA assay is to ascertain if perturbation of AFP by Compound 47 is due to direct gene regulation effect or indirect consequence of altered cell proliferation. Using HuH7 (high AFP-producing), we found AFP transcript to be significantly repressed to one-third within 24 h after Compound 47 administration (10 μM) while sunitinib showed no measurable effect (FIG. 6).

### Example 11

### Profiling RTK Targets by Antibody Array

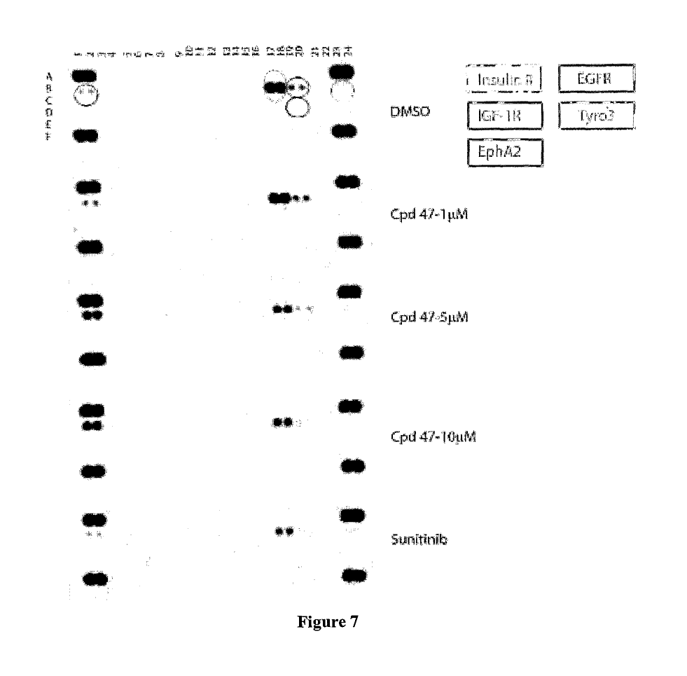

Then the RTKs targets of Compound 47 were considered for its efficacy in HCC cell lines. Profiling of receptor tyrosine kinase(s) inhibition by either Compound 47 or sunitinib was determined by incubating lysates of treated cells with human phospho-RTK array (RnD Systems). Serum-starved HuH7 cells were used because it possessed more constitutively phosphorylated RTKs than other HCC cell lines employed in this study (data not shown). Hence, this platform would allow us to detect more signaling changes upon inhibition of target kinases' phosphorylation. Here, notable inhibition of RTKs including insulin receptor, IGF-1R, Tyro3, EphA2, HER3, Met and RON by Compound 47 was observed (FIG. 7). The magnitude of these effects was determined densitometrically and found to be generally stronger than sunitinib, with the key exception of insulin receptor (Table 9).

Changes in RTK phosphorylation after treatment with Compound 47 or sunitinib (FIG. 7) were quantified and ranked according to the magnitude of expression in the untreated controls (highest to lowest).

TABLE 9
Quantification of RTK array data

| | | Phosphorylation (Arbitrary units) | | | | |
|---|---|---|---|---|---|---|
| Co- | | Cpd 47 | Cpd 47 | Cpd 47 | Cpd 47 | Sunitinib |
| ordinates | RTK | (0 μM) | (1 μM) | (5 μM) | (10 μM) | (10 μM) |
| B17, 18 | Insulin R | 0.74 | 0.75 | 0.4 | 0.38 | 0.24 |
| B19, 20 | IGF-1R | 0.19 | 0.22 | 0.13 | 0.07 | 0.09 |
| B1, 2 | EGFR | 0.15 | 0.19 | 0.44 | 0.53 | 0.13 |
| B23, 24 | Tyro-3 | 0.15 | 0.10 | 0.11 | 0.07 | 0.12 |
| D19, 20 | EphA2 | 0.07 | 0.09 | 0.05 | 0.02 | 0.04 |
| B5, 6 | Her3 | 0.05 | 0.07 | 0.04 | 0.03 | 0.04 |
| C3, 4 | Met | 0.05 | 0.08 | 0.05 | 0.02 | 0.04 |
| C5, 6 | Ron | 0.04 | 0.06 | 0.05 | 0.03 | 0.05 |

Other minor effects were observed for FGFR4 and CSF-1R but these spots were too faint for accurate quantification (FIG. 7).

### Example 12

### Confirming the Inhibition of IGF-1R, EphA2 and Tyro3 Phosphorylation by Western Blot

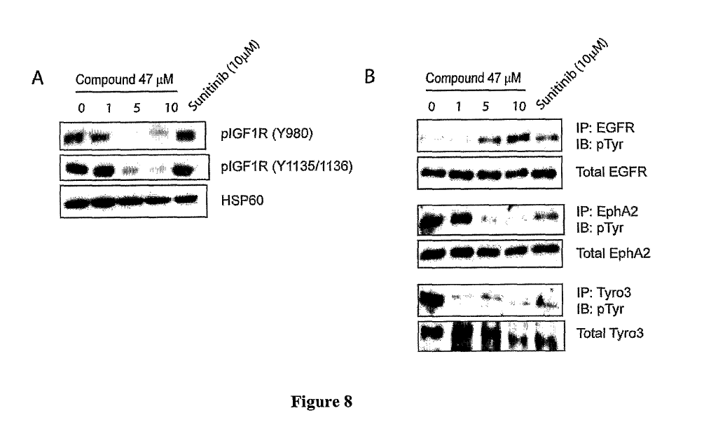

Major RTKs phosphorylation changes were determined independently by immunoblotting. Specifically, we examined the constitutively phosphorylated IGF-1R, EphA2 and Tyro3 as interesting targets which might be novel drivers for oncogenicity in HuH7. From the results, proportional reduction in IGF-1R and EphA2 phosphorylation was observed with increasing concentration of Compound 47 (FIGS. 8A and 8B). This observation was compound-specific because 10 μM sunitinib did not repress phosphorylation of either tyrosine kinases appreciably. On the other hand, Tyro3 phosphorylation was inhibited even at the lowest concentration used (1 μM) and no further depletion was observed at higher concentrations. Sunitinib displayed similar effect against Tyro3. Separately, the unexpected increase in EGFR phosphorylation was also reproduced upon treatment with Compound 47 but not sunitinib (FIG. 8B).

In summary, the inventors of the present invention found that the compounds described herein are not only suited for antiproliferative activity, but also chemoprevention. Without being bound to any particular theory, the 3′-CF₃ substituent on ring B and 6-OCH₃ substituent on ring A seemed to be important substituents for antiproliferative activity although preferences for NQO1 induction were less clear cut. A small number of compounds that combined good antiproliferative activity with chemopreventive potential (as evaluated from their NQO1/CYP1A1 quotient) were identified. These were mainly compounds (42, 45-48). These compounds had NQO1/CYP1A1 quotients that exceeded 2. The compounds 42, 45-48 are particularly promising because they have CD values in the nanomolar range but yet show at least 2-fold selectivity for NQO1 induction. These compounds could potentially have the dual effects of cytoprotection towards normal cells while being cytotoxic to cancer cells.

An unexpected finding was how structural features in the compounds with antiproliferative activity were linked to their effects on cell cycle progression. Compounds with at least two methoxy groups on rings A and B (42, 45) were found to cause G2 arrest, possibly by interfering with the formation of the mitotic spindle. By contrast, compounds with halogenated groups on both rings (46-48) caused G1 arrest which may involve inhibition of cyclin dependent kinase activity. Evidence of apoptosis was observed for these compounds.

In conclusion, the inventors have demonstrated the chemopreventive potential of the compounds described herein as seen from their ability to induce NQO1 activity. Most of the compounds evaluated had CD values that were less than 10 μM, highlighting the potential of this scaffold for chemoprevention. On the other hand, the presence of the nitrogen may have predisposed the compounds to the induction of both CYP1A1 and NQO1 activities although there were candidates that selectively induced NQO1 by 4-fold or more in this series. In the case of the antiproliferative activity, the compounds (42, 45-48) as described herein were able to successfully combine selective induction of NQO1 with good antiproliferative activity (IC₅₀<10 μM on two cancer cell lines). Thus, they are potentially useful leads for compounds that combine antiproliferative activity against cancer cells with a degree of cytoprotection mediated through NQO1 induction in normal cells.

The inventors of the present invention also screened novel benzylidene-indolinones using inhibition of cell viability in HuH7 and HepG2 as a preliminary evidence for efficacy. The indolinone ring is a versatile scaffold with multiple sites for derivatization to generate compound libraries. Importantly, this scaffold satisfies key criteria of drug-like properties such as small molecular weight (<500) for efficient delivery, and favorable lipophilicity (cLogP<5) for cellular permeability and distribution. Furthermore, since prior optimization of indolinones has also given rise to other potent multi-targeted inhibitors such as sunitinib, it was speculated that indolinones fit optimally into the chemical space of an ATP-binding pocket of multiple kinases, and that subtle functionalization of the scaffold might help uncover other analogs of clinical value.

Using this approach, the inventors of the present invention found a subset of substituted benzylidene-indolinones (Compound 46-48) being particular effectively in HCC. These molecules were exclusively 3-trifluoromethyl-substituted analogs from a compound library, hence suggesting it as a useful pharmacophore for further optimization in drug design. In particular, Compound 47 demonstrated submicromolar IC₅₀ and was superior to both sunitinib (FIG. 3), and sorafenib in HepG2 cells as reported previously (Liu L, Cao Y, Chen C, Zhang X, McNabola A, Wilkie D, et al. Sorafenib blocks the RAF/MEK/ERK pathway, inhibits tumor angiogenesis, and induces tumor cell apoptosis in hepatocellular carcinoma model PLC/PRF/5. Cancer Res 2006 Dec 15; 66(24):11851-11858). This efficacy could be extrapolated across a range of HCC cell lines of diversified etiologies. The only weak responder was the lone mesenchymal-like cell line, SK-Hep1 which has a very different pathology (secondary tumor from a distant-metastasis of colon carcinoma). Additionally, Compound 47 exhibited a hugely favorable safety profile based on a reduced cytotoxicity in THLE2. This is a particularly critical attribute for multi-targeted kinase inhibitors because the inherently low stringency in its target selectivity could inadvertently lead to more off-target effects.

As the preceding results support Compound 47 as a potential drug candidate, the kinases inhibited were retrospectively investigated and in so doing, the mechanistic basis for its activity in HuH7 were deduced (under serum-starved conditions). Arguably, such strategy would prevent a comprehensive identification of all kinases targeted. However, the benefit of this approach is to generate valuable insights on the subset of RTKs that are constitutively active in HCC and thereby, play pivotal role in sustaining tumorigenicity. Accordingly, the IGF-1R was discovered as the major target, with EphA2 and Tyro3 as novel and potentially important contributory molecules for future research in HCC pathogenesis. Interestingly, a paradoxical increase in EGFR phosphorylation was also observed. Although EGFR activation commonly correlates with increase cell proliferation through Erk signaling, such downstream effect was not detected (FIG. 5). Without wishing to be bound to any theory, one hypothesis is that EGFR activation might be a compensatory response to IGF-1R inhibition as the tumor attempts to switch oncogenic dependence to an alternative pathway. The failure of subsequent Erk phosphorylation in direct proportion with EGFR activation suggests that IGF-1R, and not EGFR, has the overriding control on tumor proliferation. The inter-dependence between EGFR and IGF-1R is supported by previous reports of cross-talk where hepatoma cells gained resistance to EGFR inhibitor, gefitinib, through IGF-1R signaling (sbois-Mouthon C, Cacheux W, Blivet-Van Eggelpoel M J, Barbu V, Fartoux L, Poupon R, et al. Impact of IGF-1R/EGFR cross-talks on hepatoma cell sensitivity to gefitinib. Int J Cancer 2006 Dec. 1; 119(1 l):2557-2566). A reciprocal inhibition of IGF-1R also displayed an increase in EGFR phosphorylation in BxPC3 cells which mirrored these observations (Buck E, Eyzaguirre A, Rosenfeld-Franklin M, Thomson S, Mulvihill M, Barr S, et al. Feedback mechanisms promote cooperativity for small molecule inhibitors of epidermal and insulin-like growth factor receptors. Cancer Res 2008 Oct. 15; 68 (20):8322-8332). Likewise, another study described that perturbation of IGF-1R directly altered ERK phosphorylation in HuH7 cells and contributed to oncogenesis (Cheng W, Tseng C J, Lin T T, Cheng I, Pan H W, Hsu H C, et al. Glypican-3-mediated oncogenesis involves the Insulin-like growth factor-signaling pathway. Carcinogenesis 2008 July; 29(7):1319-1326), thus affirming IGF-1R as the most likely transducer of MAPK signaling in HCC as compared to the more established EGFR signaling seen in other cancer types.

IGF-1R is an oncogene of increasing significance to cancer research. Reports have described the role of IGF-1R in hepatocarcinogenesis and its control over downstream cell cycling and anti-apoptotic pathways in HCC models (Cheng W, Tseng C J, Lin T T, Cheng I, Pan H W, Hsu H C, et al. Glypican-3-mediated oncogenesis involves the Insulin-like growth factor-signaling pathway. Carcinogenesis 2008 July; 29(7):1319-1326; Hopfner M, Huether A, Sutter A P, Baradari V, Schuppan D, Scherubl H. Blockade of IGF-1 receptor tyrosine kinase has antineoplastic effects in hepatocellular carcinoma cells. Biochem Pharmacol 2006 May 14; 71(10):1435-1448). The observation of IGF-1R being the most strongly phosphorylated RTK (besides the physiologically active insulin receptor) in HuH7 qualifies it as a key candidate in the maintenance of HCC phenotype. Separately, EphA2 and Tyro3 are novel oncogenes in HCC. EphA2 is an emerging target as cumulating evidences suggest its overexpression and role in several malignancies. At least in some melanoma, EphA2 activity has been demonstrated to contribute to tumor neovascularization (Walker-Daniels J, Hess A R, Hendrix M J, Kinch M S. Differential regulation of EphA2 in normal and malignant cells. Am J Pathol 2003 April; 162(4):1037-1042). Tyro3 belongs to the Ax1 subfamily of RTK. To date, limited reports associated Tyro3 to human cancers although its transformation properties have been experimentally demonstrated in experimental models (Hafizi S, Dahlback B. Gas6 and protein S. Vitamin K-dependent ligands for the Ax1 receptor tyrosine kinase subfamily. EBBS J 2006 December; 273(23):5231-5244). While the exact function of these RTKs in HCC requires further investigation, the inhibition of cell cycling markers (PCNA and Cyclin D1) and the stimulation of early apoptosis (caspase-3 activation and PARP cleavage) provided further evidence that the consequence of kinase inhibition was translated through the signaling cascade, resulting in synergistic growth arrest and cell death.

While the complete spectrum of RTK targets for Compound 47 has not been explored, the evidence provided so far illuminated the stark contrast with classical multi-RTK inhibitor, sunitinib. Insulin receptor was inhibited by Compound 47 but significantly less than with sunitinib. This may account for the reduced cytotoxicity effects observed with Compound 47 vs. sunitinib. On the contrary, the strong IGF-1R and EphA2 inhibition by Compound 47 was not seen with sunitinib. One important consequence was the marked difference in the regulation of AFP where Compound 47 dramatically depleted AFP transcription while sunitinib had negligible effects. This finding alluded to the possibility that the panel of kinase inhibited might be mechanistically regulating AFP and thereby, conferred a selective effect on HCC, where 60-70% are known to be AFP positive (Abelev G I, Eraiser T L. Cellular aspects of alpha-fetoprotein reexpression in tumors. Semin Cancer Biol 1999 April; 9(2):95-107). Moreover, a recent discovery that AFP knockdown promotes apoptosis in HuH7 cells suggests AFP may be actively regulating HCC growth instead of an innocuous tumor marker (Yang X, Zhang Y, Zhang L, Zhang L, Mao J. Silencing alpha-fetoprotein expression induces growth arrest and apoptosis in human hepatocellular cancer cell. Cancer Left 2008 Nov. 28; 271(2):281-293). Overall, the novel combination of kinase inhibition involving IGF-1R and other RTKs such as EphA2 and Tyro3 makes Compound 47 an interesting and potentially useful agent for other lead optimization and exploration of suitable RTK targets for HCC treatment.

In conclusion, new indolinone derivatives have been identified that display a therapeutic advantage towards cancer and in particularly HCC over existing TKIs. Mechanistically, IGF-1R signaling is a likely key driver for tumor sustenance in HCC, and possible roles of EphA2 and Tyro3 require further investigation. Multi-targeted kinase inhibitors centering on IGF-1R and other pathways may therefore represent a new class of TKIs for use against HCC as well as other malignancies where multiple signaling aberrations might be implicated.

One skilled in the art would also readily appreciate that the present invention is well adapted to carry out the objects and obtain the ends and advantages mentioned, as well as those inherent herein. The molecular complexes and the methods, procedures, treatments, molecules, specific compounds described herein are presently representative of preferred embodiments, are exemplary, and are not intended as limitations on the scope of the invention. Changes therein and other uses will occur to those skilled in the art which are encompassed within the spirit of the invention are defined by the scope of the claims.

It will be readily apparent to one skilled in the art that varying substitutions and modifications may be made to the invention disclosed herein without departing from the scope and spirit of the invention.

All patents and publications mentioned in the specification are indicative of the levels of those skilled in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising”, “consisting essentially of and” consisting of may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In addition, where features or aspects of the invention are described in terms of Markush groups, those skilled in the art will recognize that the invention is also thereby described in terms of any individual member or subgroup of members of the Markush group. For example, if X is described as selected from the group consisting of bromine, chlorine, and iodine, claims for X being bromine and claims for X being bromine and chlorine are fully described.

Other embodiments are within the following claims.

1-30. (canceled)

31. A pharmaceutical composition comprising a compound of formula I: 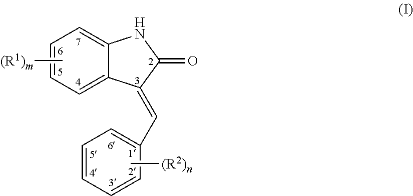
wherein, when m and n are both 1,
R¹ is one selected from the group consisting of 6-F; 6-Cl; and 6-OCH₃; and
R² is one selected from the group consisting of 3′OCH₃; 3′-OH; 4′-OCH₃; and 3′CF₃.
and, wherein when m is 1 and n is 2,
R¹ is one selected from the group consisting of 6-F; 6-Cl; and 6-OCH₃; and
R² is one selected from the group consisting of 3′OCH₃; 3′-OH; 4′-OCH₃; and
3′CF₃
or a pharmaceutically acceptable salt thereof, and a pharmaceutically acceptable carrier or excipient.

32. The pharmaceutical composition of claim 31, wherein the compound is selected from the group consisting of:
6-fluoro-3-(3′-methoxy-benzylidene)-indolin-2-one;
6-fluoro-3-(3′-hydroxy-benzylidene)-indolin-2-one;
6-fluoro-3-(4′-methoxy-benzylidene)-indolin-2-one;
6-chloro-3-(3′-methoxy-benzylidene)-indolin-2-one;
6-chloro-3-(3′-hydroxy-benzylidene)-indolin-2-one;
6-chloro-3-(4′-methoxy-benzylidene)-indolin-2-one;
6-methoxy-3-(3′-methoxy-benzylidene)-indolin-2-one;
6-methoxy-3-(3′-hydroxy-benzylidene)-indolin-2-one;
6-methoxy-3-(4′-methoxy-benzylidene)-indolin-2-one;
6-fluoro-3-(3′-hydroxy-4′-methoxy-benzylidene)-indolin-2-one;
6-chloro-3-(3′-hydroxy-4′-methoxy-benzylidene)-indolin-2-one;
6-methoxy-3-(3′-hydroxy-4′-methoxy-benzylidene)-indolin-2-one;
6-fluoro-3-(3′-trifluoromethyl-benzylidene)-indolin-2-one;
6-chloro-3-(3′-trifluoromethyl-benzylidene)-indolin-2-one; and
6-methoxy-3-(3′-trifluoromethyl-benzylidene)-indolin-2-one.

33. The pharmaceutical composition of claim 32, wherein the compound is 6-chloro-3-(3′-trifluoromethyl-benzylidene)-indolin-2-one.

34. The pharmaceutical composition of claim 31, further comprising an additional medicament or drug.

35. A compound of formula I: 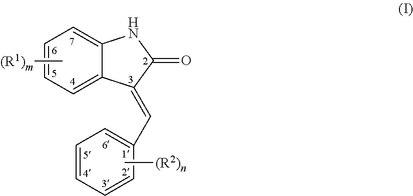
wherein the compound is selected from the group consisting of:
6-fluoro-3-(3′-methoxy-benzylidene)-indolin-2-one;
6-methoxy-3-(3′-methoxy-benzylidene)-indolin-2-one;
6-fluoro-3-(3′-hydroxy-4′-methoxy-benzylidene)-indolin-2-one;
6-chloro-3-(3′-hydroxy-4′-methoxy-benzylidene)-indolin-2-one;
6-methoxy-3-(3′-hydroxy-4′-methoxy-benzylidene)-indolin-2-one;
6-fluoro-3-(3′-trifluoromethyl-benzylidene)-indolin-2-one; and
6-methoxy-3-(3′-trifluoromethyl-benzylidene)-indolin-2-one.

36. 5-chloro-3-(3′-methoxy-benzylidene)-indolin-2-one.

37. A method of preparing a compound of formula I as defined in claim 31, the method comprising reacting an oxindole of formula (III) 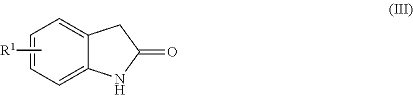
with an aldehyde of formula (IV) 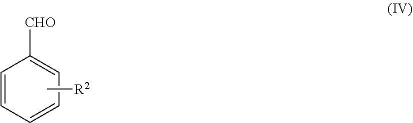
in the presence of a base.

38. A pharmaceutical composition comprising a compound or salt of claim 36 and a pharmaceutically acceptable carrier or excipient.

39. The pharmaceutical composition of claim 38, further comprising an additional medicament or drug.

40. A method for the modulation of the catalytic activity of a protein kinase comprising contacting said protein kinase with a compound of Formula I 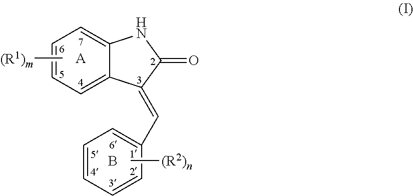
wherein, when m and n are both 1,
R¹ is one selected from the group consisting of 6-F; 6-Cl; and 6-OCH₃; and
R² is one selected from the group consisting of 3′OCH₃; 3′-OH; 4′-OCH₃; and
3′CF₃.
and, wherein when m is 1 and n is 2,
R¹ is one selected from the group consisting of 6-F; 6-Cl; and 6-OCH₃; and
R² is one selected from the group consisting of 3′OCH₃; 3′-OH; 4′-OCH₃; and
3′CF₃.

41. The method of claim 40, wherein said protein kinase comprises a protein tyrosine kinase.

42. The method of claim 40, wherein said protein tyrosine kinase comprises a receptor protein tyrosine kinase.

43. The method of claim 42, wherein said receptor protein tyrosine kinase is selected from the group consisting of EGFR, HER2, HER3, HER4, IR, IGF-1R, IRR, PDGFR CSFIR, C-Kit, C-fms, Flk-1R, F1k4, KDR/Flk1, Flt-1, FGFR-1R, FGFR-2R, FGFR-3R, FGFR-4R, EphA2 and Tyro3.

44. A method for the treatment or prevention of protein tyrosine kinase-related disease or disorder, comprising the administration of a pharmaceutically active amount of a compound of Formula I 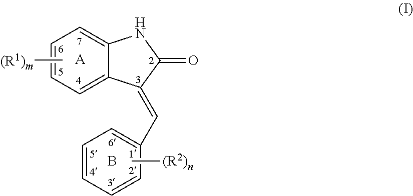
wherein, when m and n are both 1,
R¹ is one selected from the group consisting of 6-F; 6-Cl; and 6-OCH₃; and
R² is one selected from the group consisting of 3′OCH₃; 3′-OH; 4′-OCH₃; and
3′CF₃.
and, wherein when m is 1 and n is 2,
R¹ is one selected from the group consisting of 6-F; 6-Cl; and 6-OCH₃; and
R² is one selected from the group consisting of 3′OCH₃; 3′-OH; 4′-OCH₃; and
3′CF₃,
to a subject in need thereof.

45. The method of claim 44, wherein the subject is a mammal, preferably a human.

46. The method of claim 44, wherein the protein tyrosine kinase-related disease or disorder comprises a receptor protein tyrosine kinase related disorder.

47. The method of claim 44, wherein the protein tyrosine kinase-related disease or disorder comprises an IGF-1R related disorder.

48. The method of claim 47, wherein said IGF-1R related disorder is cancer.

49. The method of claim 44, wherein the protein tyrosine kinase-related disease or disorder is selected from the group consisting of hepatocellular carcinoma, breast cancer, colon cancer and lung cancer.

50. The method of claim 49, wherein the protein tyrosine kinase-related disease or disorder is hepatocellular carcinoma.

51. A method of identifying protein kinase inhibitor having specific efficacy against hepatocellular carcinoma, the method comprising:
(i) incubating a candidate compound of formula I, 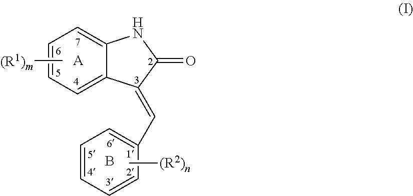
wherein, when m and n are both 1,
R¹ is one selected from the group consisting of 6-F; 6-Cl; and 6-OCH₃; and
R² is one selected from the group consisting of 3′OCH₃; 3′-OH; 4′-OCH₃; and
3′CF₃.
and, wherein when m is 1 and n is 2,
R¹ is one selected from the group consisting of 6-F; 6-Cl; and 6-OCH₃; and
R² is one selected from the group consisting of 3′OCH₃; 3′-OH; 4′-OCH₃; and
3′CF₃;
with a hepatocellular carcinoma cell line;
(ii) determining cell viability; and
(iii) comparing the determined cell viability with the candidate compound's effects on a normal liver cell line to identify a compound with specific activity against hepatocellular carcinoma cells.

52. The method of claim 51, wherein the hepatocellular carcinoma cell line is selected from the group consisting of HepG2 and HuH7.
